(19) United States
(12) Patent Application Publication
(10) Pub. No.: US 2026/0256920 A1
(43) Pub. Date: Sep. 3, 2026

(54) SELECTIVE HISTONE DEACETYLASE 3 (HDAC3) DERADERS AND METHODS OF USE THEREOF

(71) Applicant: Dana-Farber Cancer Institute, Inc., Boston, MA (US)

(72) Inventors: Eric S. FISCHER, Boston, MA (US); Yuan XIONG, Boston, MA (US); Katherine A. DONOVAN, Boston, MA (US)

(73) Assignee: Dana-Farber Cancer Institute, Inc., Boston, MA (US)

(21) Appl. No.: 18/877,720

(22) PCT Filed: Jun. 29, 2023
(86) PCT No.: PCT/US2023/026519

§ 371 Date: Dec. 20, 2024

Related U.S. Application Data
(60) Provisional application No. 63/357,045, filed on Jun. 30, 2022.

(51) Int. Cl. A61K 47/54

(52) U.S. Cl. CPC A61K 47/545

(57) ABSTRACT

The present disclosure relates to compounds comprising histone deacetylase 3 (HDAC3) targeting ligand conjugated to a Degron binding E3 ubiquitin ligase to achieve recruitment of the E3 ubiquitin ligase to HDAC3. Further disclosed compositions and methods for treating diseases or conditions mediated by aberrant HDAC3 activity comprising administering a therapeutically effective amount of the compound or the pharmaceutical composition to a subject in need thereof.

### RELATED APPLICATIONS

This application claims the benefit of priority under 35 U.S.C. § 119 (e) to U.S. Provisional Application No. 63/357,045, filed Jun. 30, 2022, which is incorporated herein by reference in its entirety.

### BACKGROUND OF THE DISCLOSURE

The modification of histones by acetylation/deacetylation plays a key role in the regulation of gene expression by changing the structure of chromatin and by modulating the accessibility of transcription factors to their target DNA sequences (Eckschlager, et al., Int. J. Mol. Sci. 18:1414 (2017)). The acetylation state of histones and other proteins is maintained by histone acetyltransferases (HAT) and histone deacetylases (HDAC). HATs add acetyl groups to lysine residues, and HDACs remove the acetyl groups. Generally, the acetylation of histone promotes a more relaxed chromatin structure which allows for transcriptional activation (Xu et al., Oncogene 26:5541-5552 (2007)). In addition to regulating histone modification, HDACs also regulate the post-translational acetylation of many non-histone proteins, including transcription factors, chaperones, and signaling molecules, resulting in changes in protein stability, protein-protein interactions, and protein-DNA interactions (Glozak, et al., Gene 363:15-23 (2005)). The balance between histone acetylation and deacetylation is usually well regulated, but the balance is often upset in diseases such as cancer and neurodegenerative diseases.

HDACs are composed of 18 members (isoforms) which are divided into 4 classes based on their homology. There are 11 conventional HDACs that require Zn²⁺ as a cofactor for their deacetylase activity; they fall within classes I, II, and IV. Class I HDACs, which include HDACs 1, 2, 3, and 8, are located only within the nucleus and are related to yeast RPD3 gene. Class II HDACs include HDACs 4, 5, 6, 7, 9, and 10 which are located in both the nucleus and the cytoplasm and are related to yeast Hda1 gene. Class IV includes HDAC 11 and has features in common with both Class I and Class II HDACs. Unlike conventional HDACs, Class III HDACs are composed of 7 mammalian sirtuins (SIRT1-7), which include nicotinamide adenine dinucleotide (NAD⁺)-dependent protein deacetylases localized in the nucleus (SIRT1, SIRT6, and SIRT7), mitochondria (SIRT3, SIRT4, and SIRT5), and cytoplasm (SIRT2) (Kim, et al., Am. J. Transl. Res. 3:166-179 (2011)).

In view of the many HDAC isoforms, HDAC inhibition has a narrow therapeutic window and an accompanying risk of causing several adverse side effects. Accordingly, there is a need for compounds that inhibit specific HDAC isoforms (e.g., HDAC3) while minimizing off-target toxicity caused by binding to other unintended HDAC isoforms, for use in treating diseases such as cancer and neurodegenerative diseases.

### SUMMARY OF THE DISCLOSURE

A first aspect of the present disclosure is directed to a compound having a structure represented by formula (I):

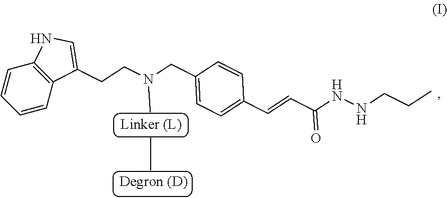

or a pharmaceutically acceptable salt or stereoisomer thereof,
wherein:
the Linker represents a moiety that connects covalently the nitrogen atom and Degron; and
the Degron is of Formula D1 or D2:

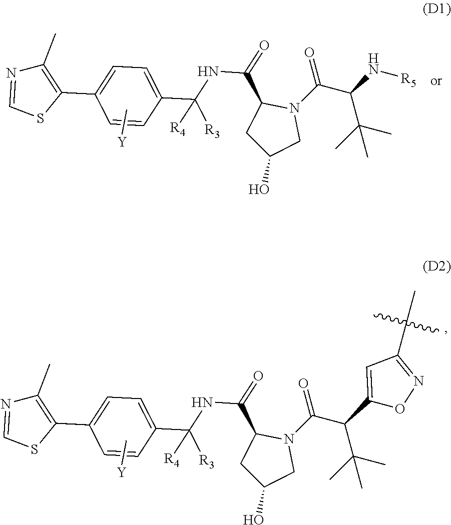

wherein:
   R₃ is hydrogen or optionally substituted C₁-C₃ alkyl, or
   R₃ and R₄, together with the carbon atom to which they are attached, form cyclopropyl;
   R₄ is hydrogen, methyl, or

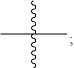

R₅ is C(O)CR₆R₇R₈,

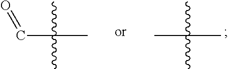

R₆ and R₇ are hydrogen, or
R₆ and R₇, together with the carbon atom to which they are attached, form cyclopropyl;
   R₈ is hydrogen, fluoro, cyano, or NMe₂; and
Y is hydrogen,

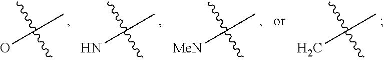

wherein

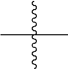

is a bond between the Degron and the Linker, provided that there is only one bond between the Degron and the Linker.

Another aspect of the present disclosure is directed to a pharmaceutical composition containing a therapeutically effective amount of a compound of formula (I) or a pharmaceutically acceptable salt or stereoisomer thereof, and a pharmaceutically acceptable carrier.

In another aspect of the present disclosure, methods of making the compounds are provided.

A further aspect of the present disclosure is directed to a method of treating a disease or disorder characterized or mediated by aberrant HDAC3 activity, that includes administering a therapeutically effective amount of a compound of formula (I) or a pharmaceutically acceptable salt or stereoisomer thereof, to a subject in need thereof.

As shown in working examples herein, compounds of formula (I) (also referred to herein as degraders) cause degradation of HDAC3 while substantially sparing other HDAC isoforms.

Accordingly, the compounds of the present disclosure may serve as a set of new chemical tools for HDAC3 knockdown, exemplify a broadly applicable approach to arrive at degraders that are selective over non-selective binding ligands, and may provide effective treatments for HDAC3-mediated diseases and disorders such as cancer (e.g., hematological cancer), neurodegenerative diseases (e.g., Parkinson's disease, Alzheimer's disease, and Huntington's disease), and autoimmune diseases.

### BRIEF DESCRIPTION OF THE DRAWINGS

FIG. 1A and FIG. 1B are scatter plots that show the relative change in relative protein abundance with treatment of Kelly cells with compounds 2 (FIG. 1A) and 3 (FIG. 1B).

FIG. 2A and FIG. 2B are plots of a cellular VHL engagement assay for compounds 1 (FIG. 2A) and 2 (FIG. 2B).

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the disclosure.

With respect to compounds of the present disclosure, and to the extent the following terms are used herein to further describe them, the following definitions apply.

As used herein, the term “alkyl” refers to a saturated linear or branched-chain monovalent hydrocarbon radical. In some embodiments, the alkyl radical is a C₁-C₆ group. In some embodiments, and to the extent not disclosed otherwise for any one or more groups of the compounds of formula (I), the alkyl radical is a C₀-C₆, C₀-C₅, C₀-C₃, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. In some embodiments, an alkyl group is a C₁-C₃ alkyl group. In some embodiments, an alkyl group is a C₁-C₂ alkyl group. In some embodiments, an alkyl group is a methyl group.

As used herein, the term “alkylene” refers to a straight or branched divalent hydrocarbon chain linking the rest of the molecule to a radical group, consisting solely of carbon and hydrogen, containing no unsaturation and having from one to 15 carbon atoms, for example, methylene, ethylene, propylene, n-butylene, and the like. The alkylene chain may be attached to the rest of the molecule through a single bond and to the radical group through a single bond. In some embodiments, the alkylene group contains one to 15 carbon atoms (C₁-C₁₅ alkylene). In some embodiments, the alkylene group contains one to 12 carbon atoms (C₁-C₁₂ alkylene). In some embodiments, the alkylene group contains one to 10 carbon atoms (C₁-C₁₀ alkylene). In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some embodiments, the alkenyl radical is a C₂-C₁₅ group. In some embodiments, and to the extent not disclosed otherwise for any one or more groups of the compounds of formula (I), the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In some embodiments, the alkynyl radical is a C₂-C₁₅ group. In some embodiments, and to the extent not disclosed otherwise for any one or more groups of the compounds of formula (I), the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. In some embodiments, the alkoxyl group is methoxy, ethoxy, propyloxy, or tert-butoxy. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 12 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 10 carbon atoms (C₃-C₁₀). In one embodiment, carbocyclyl includes 3 to 6 carbon atoms (C₃-C₆). In one embodiment, carbocyclyl includes 5 to 6 carbon atoms (C₅-C₆). In some embodiments, carbocyclyl, as a bicycle, includes C₆-C₁₀. In another embodiment, carbocyclyl, as a spiro system, includes C₅-C₁₁. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and phenyl; bicyclic carbocyclyls having 7 to 11 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R_{c}-carbocyclyl where R_{c} is an alkylene chain. The term carbocyclic also embraces carbocyclylalkoxy groups which as used herein refer to a group bonded through an oxygen atom of the formula —O—R_{c}-carbocyclyl where R_{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, biphenyl, 1,2,3,4-tetrahydronaphthalenyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R_{c}-aryl where R_{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R_{c}-aryl where R_{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, 4, or 5) carbon atoms have been replaced with a heteroatom or heteroatom-containing group (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3- to 12-membered heterocyclyl ring system. In some embodiments, a heterocyclyl refers to a saturated ring system, such as a 3- to 12-membered saturated heterocyclyl ring system. In some embodiments, a heterocyclyl refers to a heteroaryl ring system, such as a 5- to 12-membered heteroaryl ring system. The term heterocyclyl also includes C₂-C₈ heterocycloalkyl, which is a saturated or partially unsaturated mono-, bi-, or spiro-ring system containing 2-8 carbons and one or more (e.g., 1, 2, or 3) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from O, N, and S. In some embodiments, heterocyclyl includes 4- to 6-membered monocycles having one or more heteroatoms selected from O, N, and S. In some embodiments, heterocyclyl includes 3-membered monocycles. In some embodiments, heterocyclyl includes 4-membered monocycles. In some embodiments, heterocyclyl includes 5- to 6-membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be substituted (e.g., methyl, isopropyl) and/or quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-8-azabicyclo[3.2.1]octanyl, 2-azabicyclo[2.2.2]octanyl, 8-azabicyclo[3.2.1]octanyl, azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl (e.g., thiazol-2-yl), thiadiazolyl (e.g., 1,3,4-thiadiazol-5-yl and 1,2,4-thiadiazol-5-yl), oxazolyl (e.g., oxazol-2-yl), and oxadiazolyl (e.g., 1,3,4-oxadiazol-5-yl and 1,2,4-oxadiazol-5-yl). Example of 5-membered heterocyclyls containing 2 to 4 nitrogen atoms include imidazolyl (e.g., imidazol-2-yl), triazolyl (e.g., 1,3,4-triazol-5-yl, 1,2,3-triazol-5-yl, and 1,2,4-triazol-5-yl), and tetrazolyl (e.g., 1H-tetrazol-5-yl). Representative examples of benzo-fused 5-membered heterocyclyls include benzoxazol-2-yl, benzthiazol-2-yl and benzimidazol-2-yl. Example of 6-membered heterocyclyls containing one to three nitrogen atoms and optionally a sulfur or oxygen atom are pyridyl (e.g., pyrid-2-yl, pyrid-3-yl, and pyrid-4-yl), pyrimidyl (e.g., pyrimid-2-yl and pyrimid-4-yl), triazinyl (e.g., 1,3,4-triazin-2-yl and 1,3,5-triazin-4-yl), pyridazinyl (e.g., pyridazin-3-yl), and pyrazinyl. In some embodiments, a heterocyclic group includes a heterocyclic ring fused to one or more (e.g., 1 or 2) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen atom and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, 1-pyrazolidinyl, 1-imidazolinyl and 1-imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2- or 3-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R_{c}-heterocyclyl where R_{c} is an alkylene chain.

The term heterocyclic also embraces heterocyclylalkoxy groups which as used herein refer to a radical bonded through an oxygen atom of the formula —O—R_{c}-heterocyclyl where R_{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”)) refers to a monocyclic, bicyclic or tricyclic ring system having 5 to 12 ring atoms, wherein at least one ring is aromatic and contains at least one heteroatom. In one embodiment, heteroaryl includes 5- to 6-membered monocyclic aromatic groups where one or more ring atoms is O, N, or S. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, and 1,2,3-triazol-5-yl. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3 (4H)-one. A heteroaryl group may be mono-, bi- or tri-cyclic. In some embodiments, a heteroaryl group includes a heteroaryl ring fused to one or more (e.g., 1 or 2) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R_{c}-heteroaryl, wherein R_{c} is an alkylene chain as defined above. The term heteroaryl also embraces heteroaralkoxy (or heteroarylalkoxy) groups which as used herein refer to a group bonded through an oxygen atom of the formula —O—R_{c}-heteroaryl, where Rₑ is an alkylene group as defined above.

Unless stated otherwise, and to the extent not further defined for any particular group(s) in the compounds of formula (I), any of the groups described herein may be substituted or unsubstituted. To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3- to 12-membered, 5- to 6-membered), substituted heterocyclic (e.g., substituted 3- to 12-membered, 5- to 6-membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the compounds of formula (I) and the targeted protein, which in this disclosure is histone deacetylase 3 (HDAC3), via the HDAC3 targeting ligand, typically refers to an inter-molecular interaction that is preferential (also referred to herein as “selective”) in that binding of the compounds of formula (I) with other proteins present in the cell, including other HDAC isoforms, is substantially less and may be functionally insignificant. The terms “selective” and “selectivity” refer to the ability of the compound to discriminate between and among molecular targets. A selective HDAC3 degrader described herein “substantially degrades HDAC3 and “substantially spares other HDAC isoforms” in that it may have a DC50 (half maximal degradation concentration) for HDAC3 activity that is at least about 1, 2, 3, 4, 5, 6, 7, 8, 9, or 10-fold lower than the DC50 for one or more of HDAC1, HDAC2, HDAC4, HDAC5, HDAC6, HDAC7, HDAC8, HDAC9, and/or HDAC10. Thus, even though various compounds of the present disclosure may exhibit non-negligible binding other HDAC proteins, they cause selective degradation of HDAC3.

The term “binding” as it relates to interaction between the degron and a E3 ubiquitin ligase, which in this disclosure includes von Hippel-Lindau (VHL) tumor suppressor, typically refers to an inter-molecular interaction that may or may not exhibit an affinity level that equals or exceeds that affinity between the compound and HDAC3, but is sufficient nonetheless to achieve recruitment of the E3 ubiquitin ligase to HDAC3.

Broadly, the compounds of the disclosure are represented by formula (I):

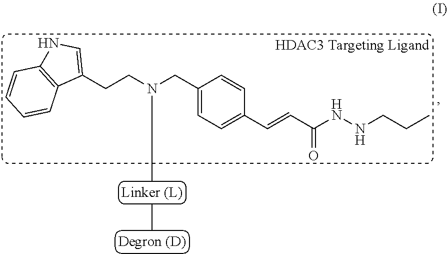

or a pharmaceutically acceptable acceptable salt or stereoisomer thereof,
wherein:
   the Linker represents a moiety that connects covalently the nitrogen atom and Degron; and
   the Degron is of Formula D1 or D2:

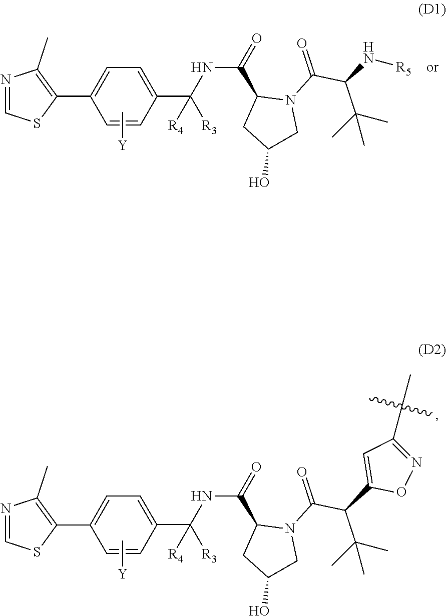

wherein:
   R₃ is hydrogen or optionally substituted C₁-C₃ alkyl, or
   R₃ and R₄, together with the carbon atom to which they are attached, form cyclopropyl;
   R₄ is hydrogen, methyl, or

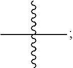

R₅ is C(O)CR₆R₇R₈,

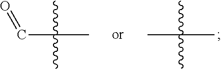

R₆ and R₇ are hydrogen, or
R₆ and R₇, together with the carbon atom to which they are attached, form cyclopropyl;
R₈ is hydrogen, fluoro, cyano, or NMe₂; and

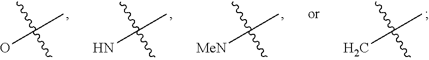

Y is hydrogen,
wherein

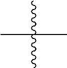

is a bond between the Degron and the Linker, provided that there is only one bond between the Degron and the Linker.

### Linkers

The linker (“L”) provides a covalent attachment between the targeting ligand and the degron.

In some embodiments, the linker is of formula L0:

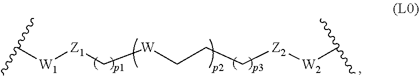

or stereoisomer thereof, wherein
p1 is an integer selected from 0 to 6;
p2 is an integer selected from 0 to 12;
p3 is an integer selected from 0 to 12;
each W is independently absent, CH₂, O, S, NR₁₀, or C(O)NR₁₀;
each R₁₀ is independently hydrogen or C₁-C₆ alkyl;
W₁ and W₂ are independently absent, (CH₂)₁₋₃, O, or NH; and
Z₁ and Z₂ are independently absent, —O—, —S—, —N(R₁₀)—, —C≡C—, —C(O)—, —C(O)O—, —OC(O)—, —OC(O)O—, C(NOR₁₀)—, —C(O)N(R₁₀)—, —C(O)N(R₁₀)C(O)—, —C(O)N(R₁₀) C(O)N(R₁₀)—, —N(R₁₀)C(O)—, —N(R₁₀) C(O)N(R₁₀)—, —N(R₁₀)C(O)O—, —OC(O)N(R₁₀)—, —C(NR₁₀)—, —N(R₁₀)C(NR₁₀)—, —C(NR₁₀)N(R₁₀)—, —N(R₁₀)C(NR₁₀)N(R₁₀)—, —OB(Me)O—, —S(O)₂—, —OS(O)—, —S(O)O—, —S(O), —OS(O)₂—, —S(O)₂O—, —N(R₁₀)S(O)₂, —S(O)₂N(R₁₀)—, —N(R₁₀)S(O)—, —S(O)N(R₁₀)—, —N(R₁₀)S(O)₂N(R₁₀)—, —N(R₁₀)S(O)N(R₁₀)—, C₃-C₁₂ carbocyclene, 3- to 12-membered heterocyclene, or 5- to 12-membered heteroarylene;
wherein the Linker is covalently bonded to a Degron via the

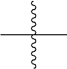

next to W₂, and covalently bonded to a Targeting Ligand via the

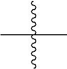

next to W₁, or the Linker is covalently bonded to a Degron via the

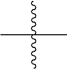

next to W₁, and covalently bonded to a Targeting Ligand via the

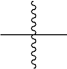

next to W₂.

In some embodiments, formula L0 is of formula L0a-L0h:

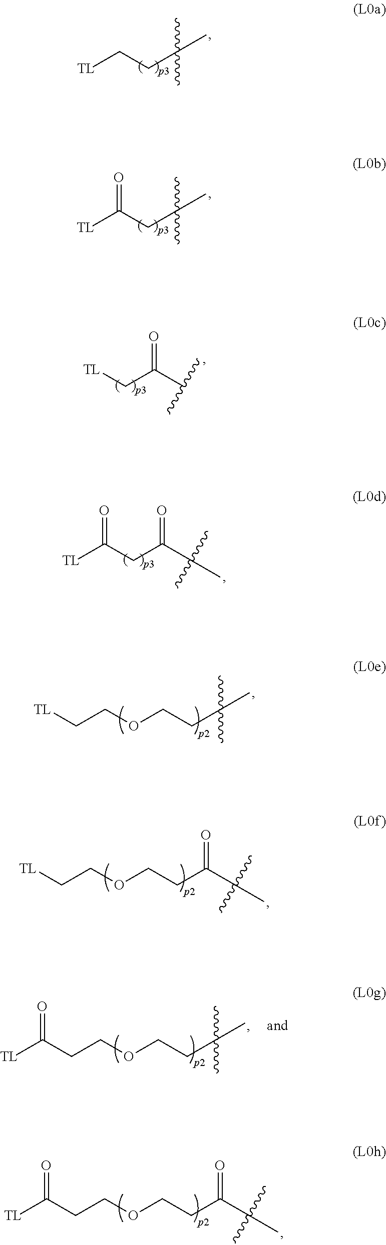

wherein TL represents targeting ligand.

In some embodiments, the linker is a bond or comprises an alkylene chain (e.g., having 2-20 alkylene units) or a bivalent alkylene chain, either of which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)—, —S(O), OS(O)₂—, —S(O)₂O—, —N(R′)S(O)₂—, —S(O)₂N(R′)—, —N(R′) S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

In some embodiments, the linker includes an alkylene chain having 1-15 alkylene units that is interrupted by and/or terminating in C(O). In some embodiments, the linker includes an alkylene chain having 1-10 alkylene units that is interrupted by and/or terminating in C(O). In some embodiments, the linker includes an alkylene chain having 1-6 alkylene units that is interrupted by and/or terminating in C(O). In some embodiments, the linker includes an alkylene chain having 1-15 alkylene units. In some embodiments, the linker includes an alkylene chain having 1-10 alkylene units. In some embodiments, the linker includes an alkylene chain having 1-6 alkylene units.

Representative examples of alkylene linkers that may be suitable for use in the compounds of the present disclosure include the following:

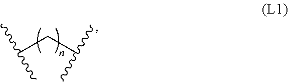

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

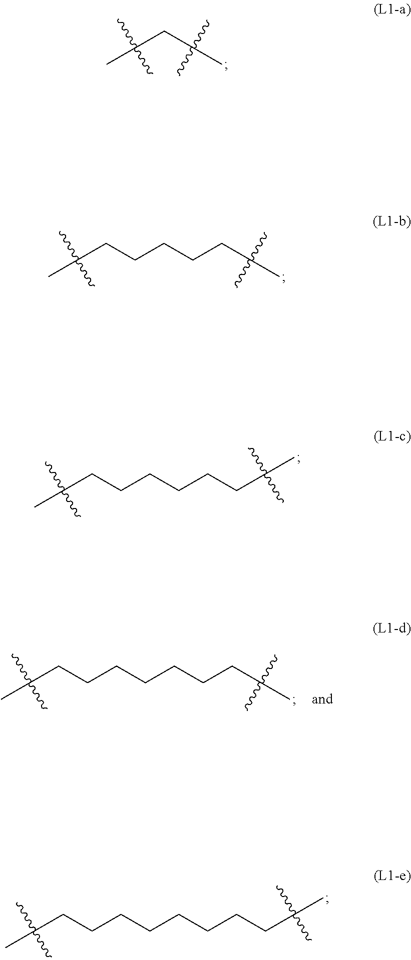

alkylene chains terminating in various functional groups (as described above), examples of which are as follows:

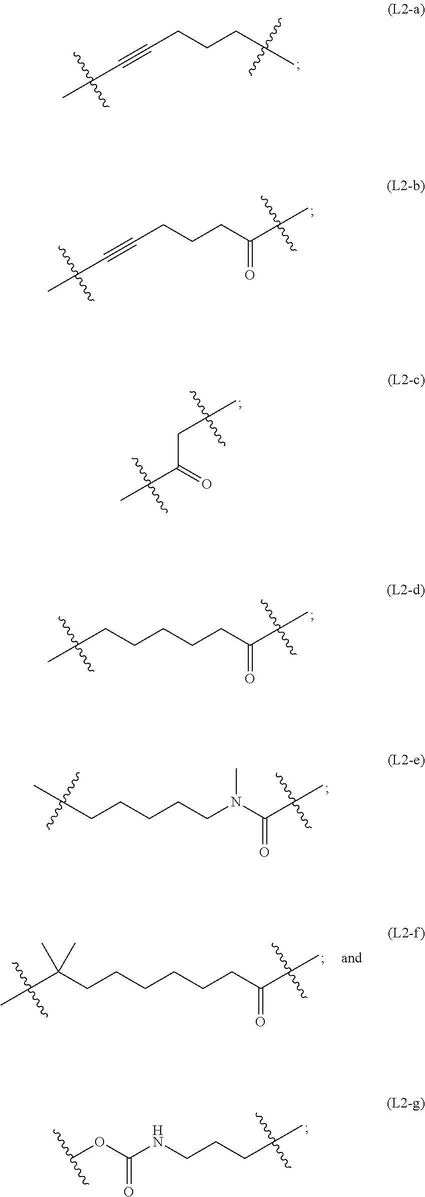

alkylene chains interrupted with various functional groups (as described above), examples of which are as follows:

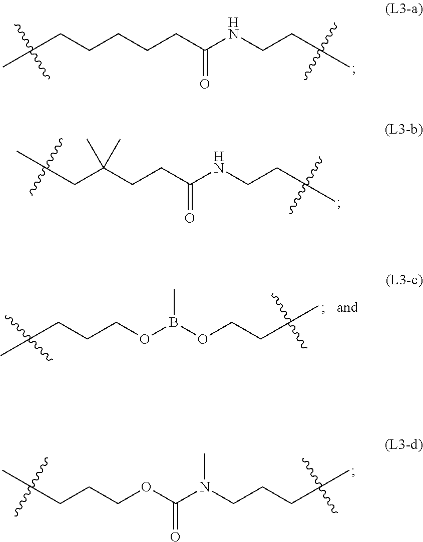 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with a heterocyclene group, e.g.,

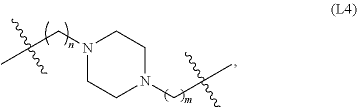

wherein m and n are independently integers of 0-10, examples of which include:

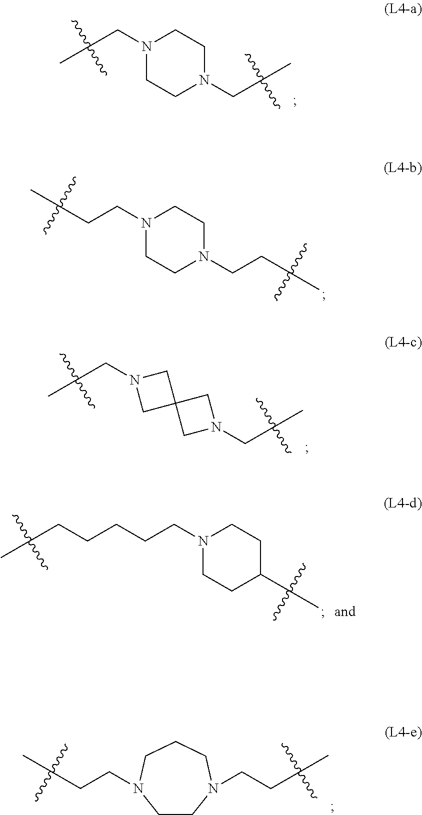 [SMILES: *CN1CCN(C*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by an amide, a heterocyclene and/or an aryl group, examples of which include:

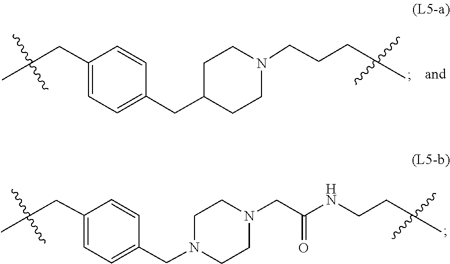 [SMILES: *CCCN1CCC(Cc2ccc(CC*)cc2)CC1 ; *CCNC(=O)CN1CCN(Cc2ccc(CC*)cc2)CC1]

alkylene chains interrupted by a heterocyclene, an aryl group, and a heteroatom, examples of which include:

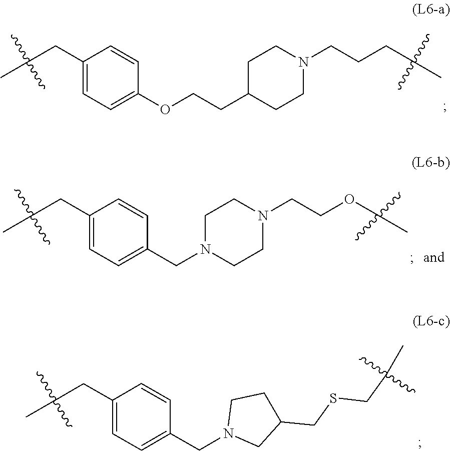

and
alkylene chains interrupted by a heteroatom such as N, O or B, e.g.,

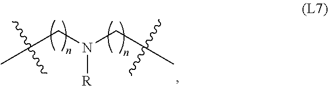

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

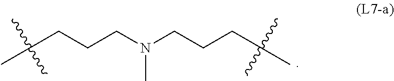 [SMILES: *CCCN(C)CCC*]

In some embodiments, the linker is a polyethylene glycol (PEG) chain which may be interrupted by, and/or terminates at either or both termini with at least one of —O—, —S—, —N(R′)—, C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′) S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker includes a polyethylene glycol chain having 1-5 PEG units and terminates in C(O). In some embodiments, the linker includes a polyethylene glycol chain having 1-5 PEG units.

Representative examples of linkers that include a polyethylene glycol chain include:

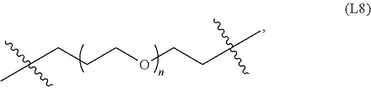

wherein n is an integer of 2-10, examples of which include:

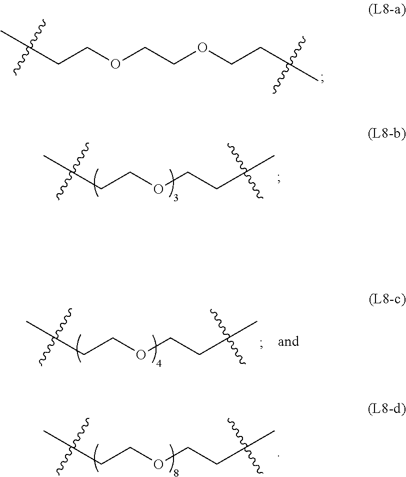

In some embodiments, the polyethylene glycol linker may terminate in a functional group, examples of which are as follows:

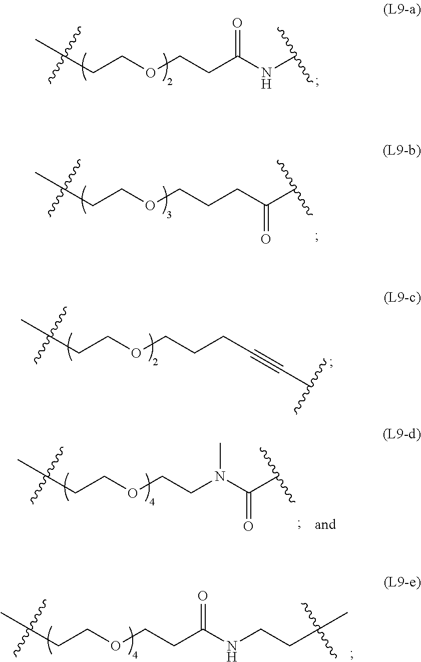

In some embodiments, the linker is represented by any one of structures:

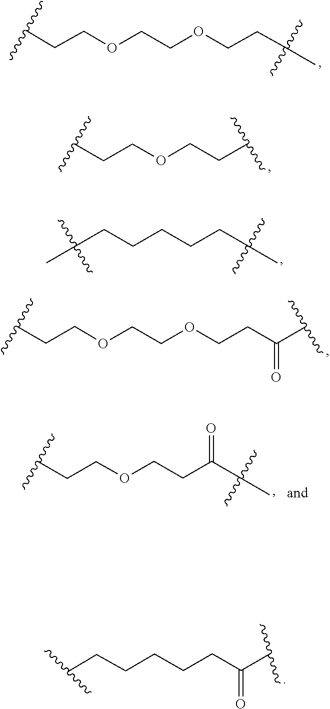 [SMILES: *CCOCCOCC* ; *CCOCC* ; *CCCCC* ; *CCOCCOCCC(*)=O ; *CCOCCC(*)=O ; *CCCCCC(*)=O]

Therefore, in some embodiments, compounds of the present disclosure may be represented by any one of the following structures:

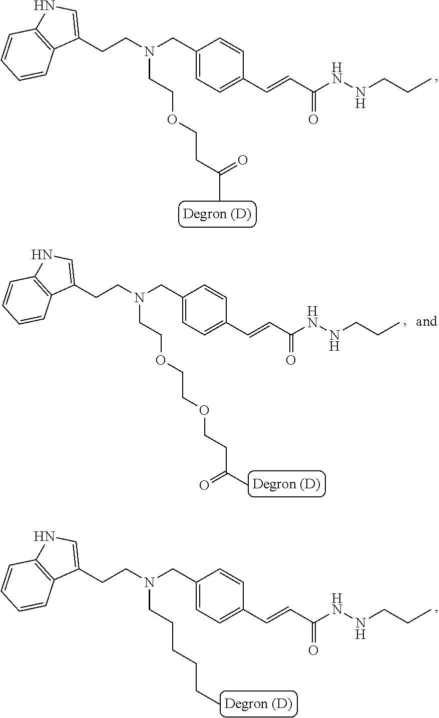 [SMILES: CCCNNC(=O)/C=C/c1ccc(CN(CCOCCC(C)=O)CCc2c[nH]c3ccccc23)cc1 ; CCCNNC(=O)/C=C/c1ccc(CN(CCOCCOCCC(C)=O)CCc2c[nH]c3ccccc23)cc1 ; CCCCCCN(CCc1c[nH]c2ccccc12)Cc1ccc(/C=C/C(=O)NNCCC)cc1]

or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

The degron may bind the E3 ligase which is von Hippel-Lindau (VHL) tumor suppressor.

Representative examples of such degrons are represented by D1 or D2:

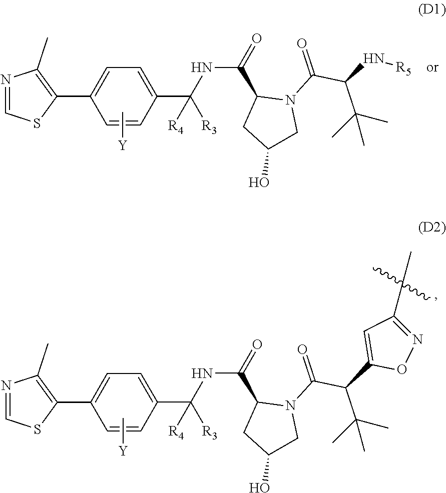

wherein:
   R₃ is hydrogen or optionally substituted C₁-C₃ alkyl, or
   R₃ and R₄, together with the carbon atom to which they are attached, form cyclopropyl;
   R₄ is hydrogen, methyl, or

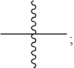

R₅ is C(O)CR₆R₇R₈,

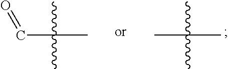

R₆ and R₇ are hydrogen, or
R₆ and R₇, together with the carbon atom to which they are attached, form cyclopropyl;
R₈ is hydrogen, fluoro, cyano, or NMe₂; and
Y is hydrogen,

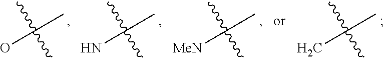

wherein

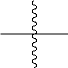

is a bond between the degron and the linker, provided that there is only one bond between the degron and the linker.

In some embodiments, the degron is of formula D1.

In some embodiments, R₃ is hydrogen and R₄ is

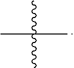

In some embodiments, R₃ and R₄ are hydrogen.

In some embodiments, R₃ is hydrogen and R₄ is methyl.

In some embodiments, R₃ is optionally substituted C₁-C₃ alkyl and R₄ is hydrogen.

In some embodiments, R₃ is optionally substituted C₁-C₃ alkyl and R₄ is methyl.

In some embodiments, R₃ and R₄, together with the carbon atom to which they are attached, form cyclopropyl.

In some embodiments, Y is hydrogen.

In some embodiments, Y is

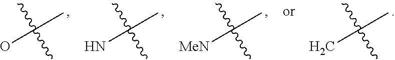

In some embodiments R₅ is

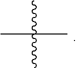

In some embodiments, R₅ is C(O)CR₆R₇R₈. In some embodiments, R₆, R₇, and R₈ are hydrogen. In some embodiments, R₆ and R₇, together with the carbon atom to which they are attached, form cyclopropyl and R₈ is hydrogen, fluoro, cyano, NMe₂. In some embodiments, R₈ is fluoro, cyano, or NMe₂.

In some embodiments, R₃ is hydrogen, R₄ is

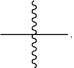

and Y is hydrogen.

In some embodiments, R₃ is hydrogen, R₄ is

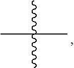

Y is hydrogen, and R₅ is C(O)CR₆R₇R₈, wherein R₆ and R₇, together with the carbon atom to which they are attached, form cyclopropyl and R₈ is fluoro, cyano, or NMe₂.

In some embodiments, Y is

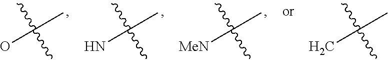

and R₃ and R₄ are hydrogen.

In some embodiments, Y is

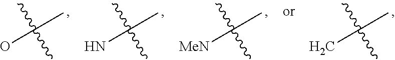

R₃ and R₄ are hydrogen, and R₅ is C(O)CR₆R₇R₈, wherein R₆ and R₇, together with the carbon atom to which they are attached, form cyclopropyl and R₈ is fluoro, cyano, or NMe₂.

In some embodiments R₅ is

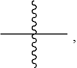

Y is hydrogen, and R₃ and R₄ are hydrogen.

In some embodiments R₅ is

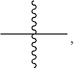

Y is hydrogen, R₃ is hydrogen, and R₄ is methyl.

In some embodiments R₅ is

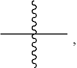

Y is hydrogen, R₃ is optionally substituted C₁-C₃ alkyl, and R₄ is hydrogen.

In some embodiments R₅ is

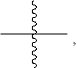

Y is hydrogen, R₃ is optionally substituted C₁-C₃ alkyl, and R₄ is methyl.

In some embodiments R₅ is

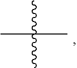

Y is hydrogen, and R₃ and R₄, together with the carbon atom to which they are attached, form cyclopropyl.

In some embodiments, formula D1 is of formula D1a-D1o:

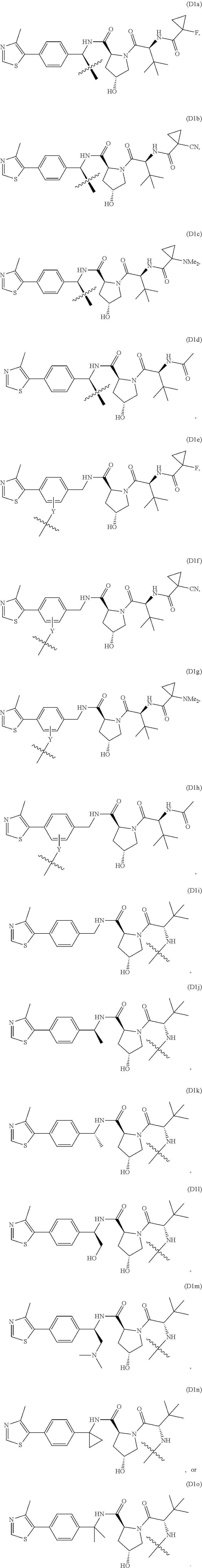

In some embodiments, the degron is of formula D2.

In some embodiments, formula D2 is of formula D2a-D2g:

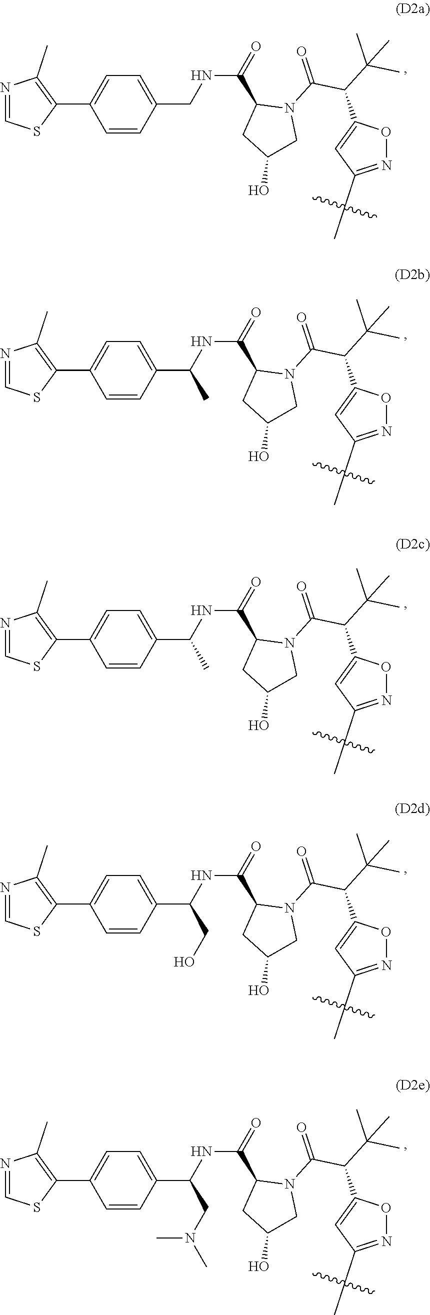 [SMILES: *c1cc([C@H](C(=O)N2C[C@H](O)C[C@H]2C(=O)NCc2ccc(-c3scnc3C)cc2)C(C)(C)C)on1 ; *c1cc([C@H](C(=O)N2C[C@H](O)C[C@H]2C(=O)N[C@@H](C)c2ccc(-c3scnc3C)cc2)C(C)(C)C)on1 ; *c1cc([C@H](C(=O)N2C[C@H](O)C[C@H]2C(=O)N[C@H](C)c2ccc(-c3scnc3C)cc2)C(C)(C)C)on1 ; *c1cc([C@H](C(=O)N2C[C@H](O)C[C@H]2C(=O)N[C@@H](CO)c2ccc(-c3scnc3C)cc2)C(C)(C)C)on1 ; *c1cc([C@H](C(=O)N2C[C@H](O)C[C@H]2C(=O)N[C@@H](CN(C)C)c2ccc(-c3scnc3C)cc2)C(C)(C)C)on1]

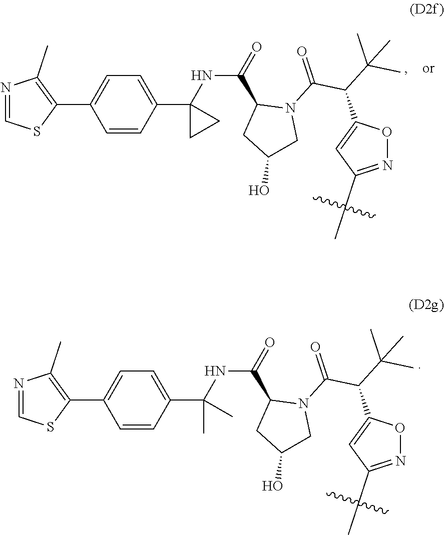 [SMILES: *c1cc([C@H](C(=O)N2C[C@H](O)C[C@H]2C(=O)NC2(c3ccc(-c4scnc4C)cc3)CC2)C(C)(C)C)on1 ; *c1cc([C@H](C(=O)N2C[C@H](O)C[C@H]2C(=O)NC(C)(C)c2ccc(-c3scnc3C)cc2)C(C)(C)C)on1]

Therefore, in some embodiments, the compounds of the present disclosure may be represented by any of the following structures:

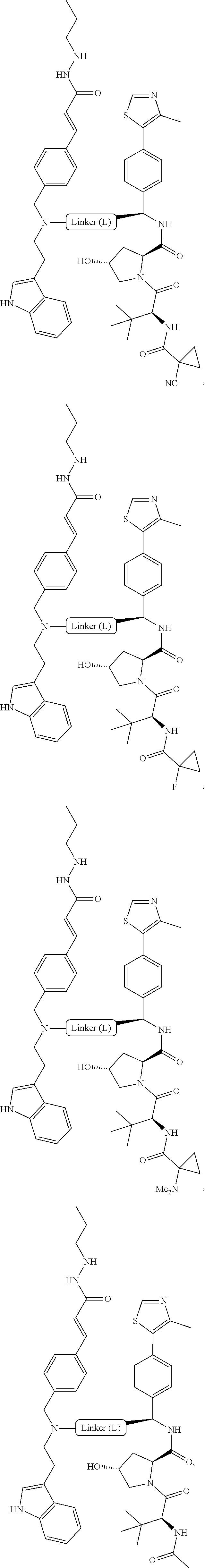

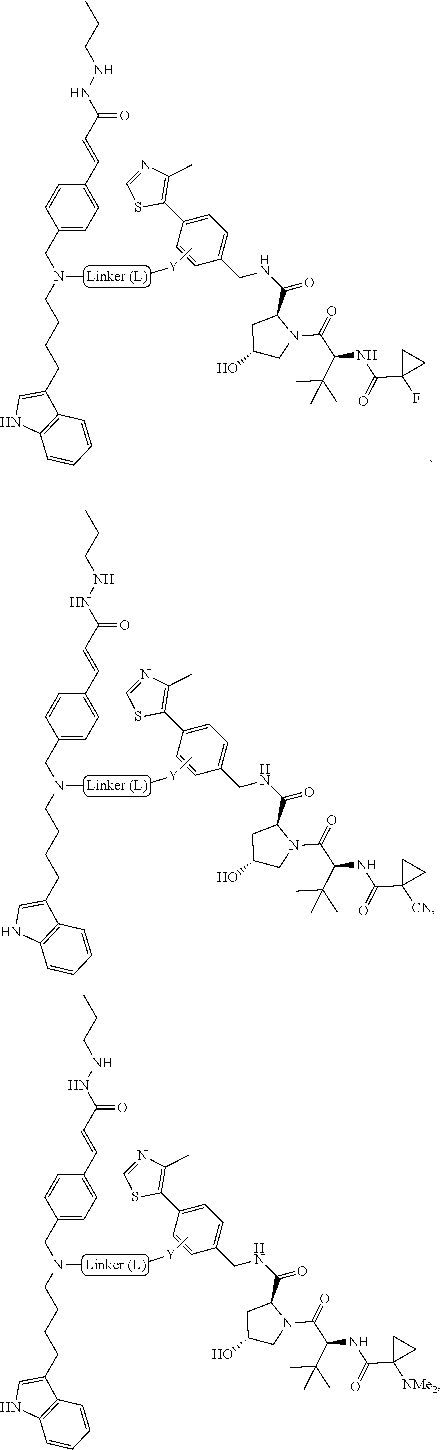

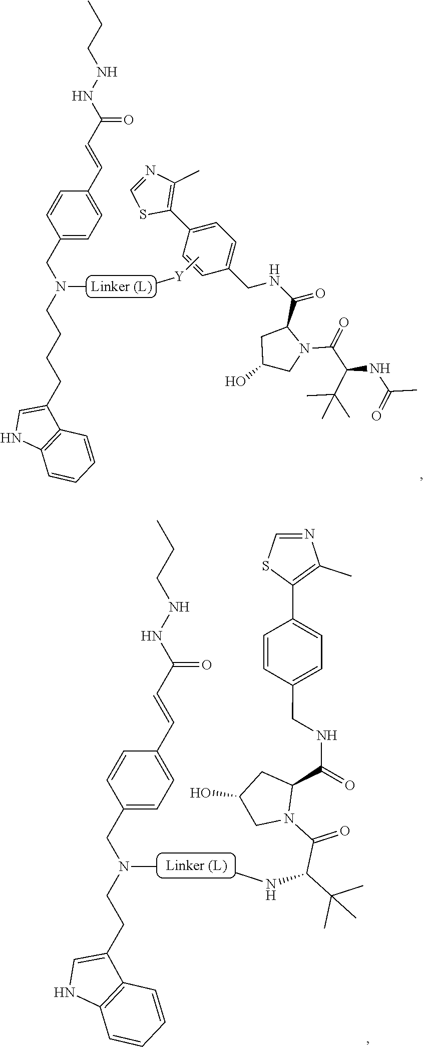

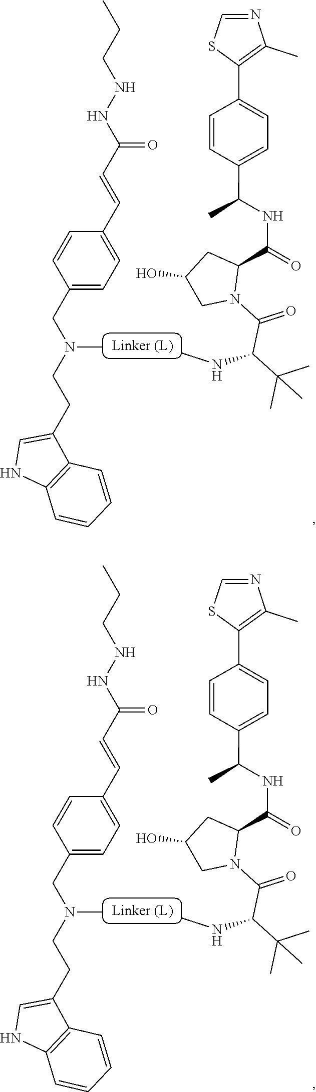

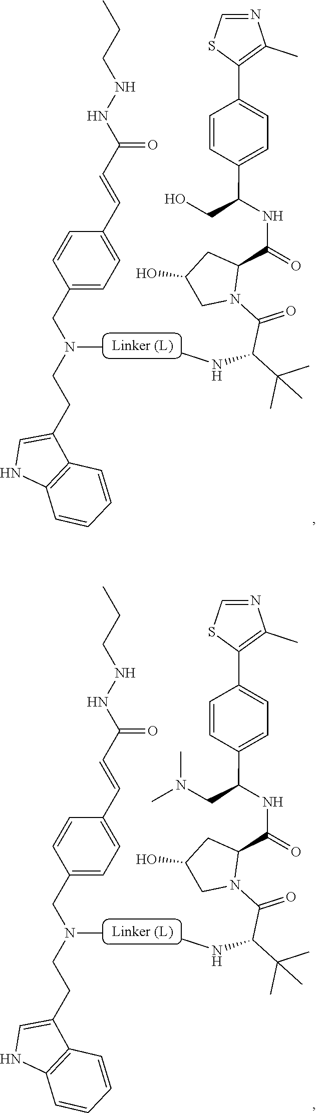

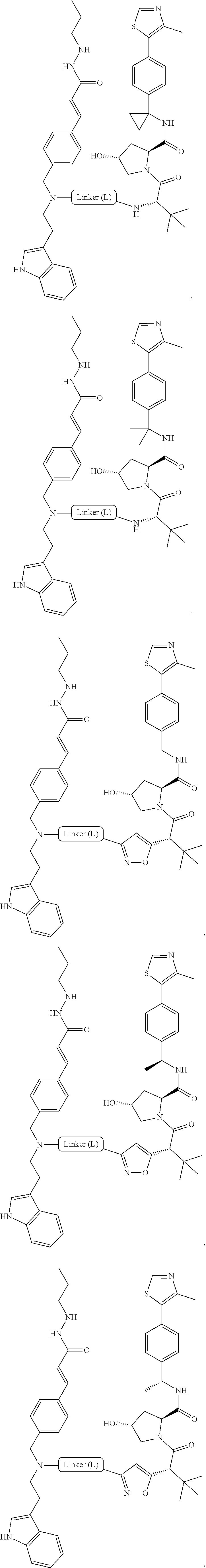

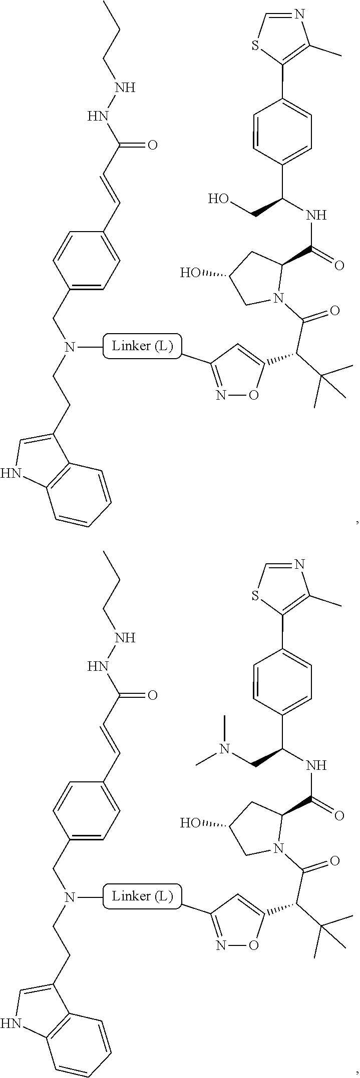

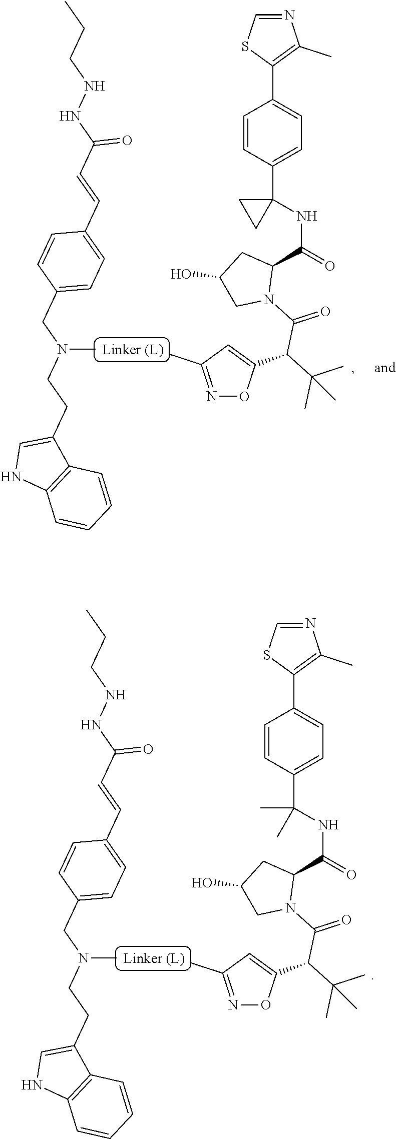 [SMILES: CCCNNC(=O)/C=C/c1ccc(CN(C)CCc2c[nH]c3ccccc23)cc1 ; Cc1cc([C@H](C(=O)N2C[C@H](O)C[C@H]2C(=O)NC2(c3ccc(-c4scnc4C)cc3)CC2)C(C)(C)C)on1 ; CCCNNC(=O)/C=C/c1ccc(CN(C)CCc2c[nH]c3ccccc23)cc1 ; Cc1cc([C@H](C(=O)N2C[C@H](O)C[C@H]2C(=O)NC(C)(C)c2ccc(-c3scnc3C)cc2)C(C)(C)C)on1]

or a pharmaceutically acceptable salt or stereoisomer thereof.

Yet other degrons that bind VHL and which may be suitable for use in the present disclosure are disclosed in U.S. Patent Application Publication 2017/0121321 A1, which is incorporated herein by reference in its entirety.

In some embodiments, compounds of the present disclosure are represented by any one of the following structures:

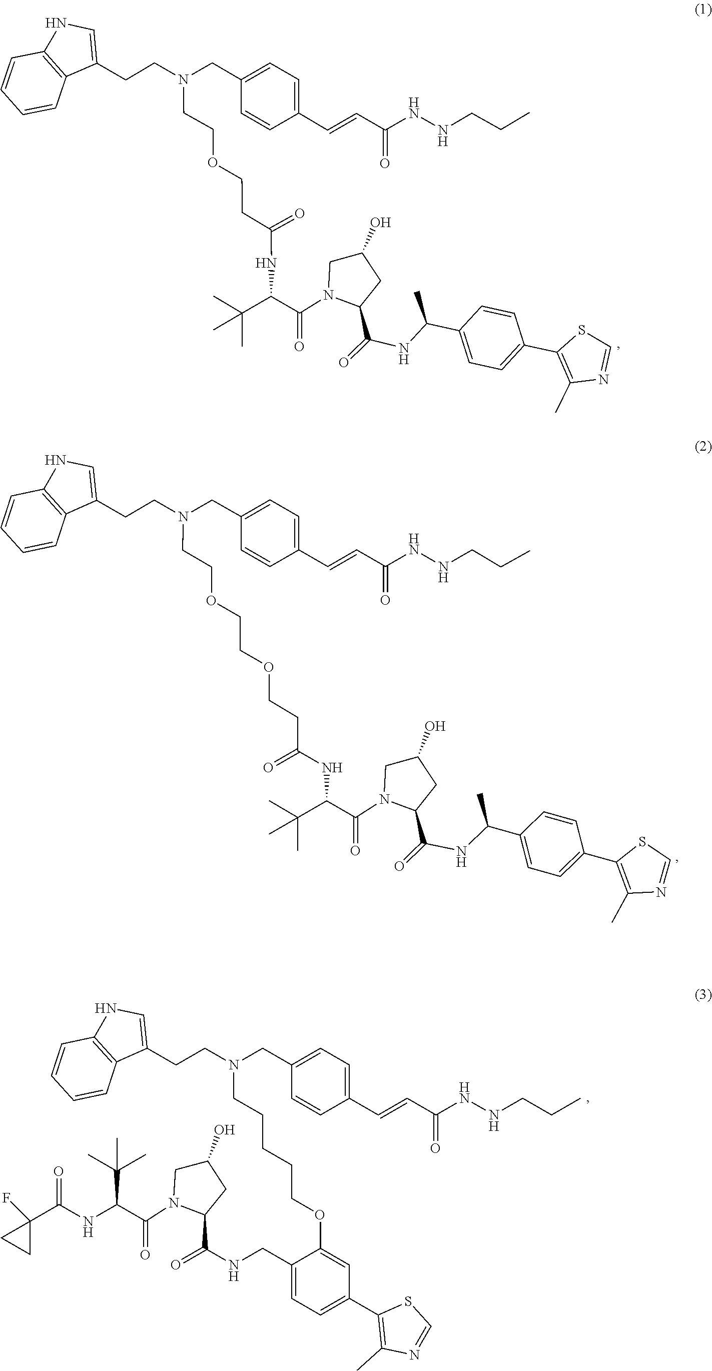 [SMILES: CCCNNC(=O)/C=C/c1ccc(CN(CCOCCC(=O)N[C@H](C(=O)N2C[C@H](O)C[C@H]2C(=O)N[C@@H](C)c2ccc(-c3scnc3C)cc2)C(C)(C)C)CCc2c[nH]c3ccccc23)cc1 ; CCCNNC(=O)/C=C/c1ccc(CN(CCOCCOCCC(=O)N[C@H](C(=O)N2C[C@H](O)C[C@H]2C(=O)N[C@@H](C)c2ccc(-c3scnc3C)cc2)C(C)(C)C)CCc2c[nH]c3ccccc23)cc1 ; CCCNNC(=O)/C=C/c1ccc(CN(CCCCCOc2cc(-c3scnc3C)ccc2CNC(=O)[C@@H]2C[C@@H](O)CN2C(=O)[C@@H](NC(=O)C2(F)CC2)C(C)(C)C)CCc2c[nH]c3ccccc23)cc1]

or pharmaceutically acceptable salt or stereoisomer thereof.

Compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present disclosure with a suitable acid or a base. Examples of pharmaceutically acceptable salts of the compounds of this disclosure include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the disclosure can form pharmaceutically acceptable salts with various organic bases such as lysine, arginine, guanidine, diethanolamine or metformin.

Compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound of formula (I) is an isotopic derivative in that it has at least one desired isotopic substitution of an atom, at an amount above the natural abundance of the isotope, i.e., enriched.

In addition, compounds of formula (I) embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In some embodiments, the present disclosure is directed to a method for making a compound of formula (I) or a pharmaceutically acceptable salt or stereoisomer thereof. Broadly, the compounds or pharmaceutically acceptable salts or stereoisomers thereof, may be prepared by any process known to be applicable to the preparation of chemically related compounds. The compounds of the present disclosure will be better understood in connection with the synthetic schemes that described in various working examples that illustrate non-limiting methods by which the compounds of the disclosure may be prepared.

### Pharmaceutical Compositions

Another aspect of the present disclosure is directed to a pharmaceutical composition that includes a therapeutically effective amount of a compound of formula (I), or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York), each of which is incorporated herein by reference in its entirety. The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal).

In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally by injection of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997), each of which is incorporated herein by reference in its entirety. Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I), or a pharmaceutically acceptable salt or a stereoisomer thereof; or a composition including a compound of formula (I), or a pharmaceutically acceptable salt or a stereoisomer thereof, effective in producing the desired therapeutic response in a particular patient in need thereof. Therefore, the term “therapeutically effective amount” includes the amount of a compound of formula (I), or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer, autophagy-dependent disease (e.g., neurodegenerative disorder)) cells, or reduces the amount of HDAC3 in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's The Pharmacological Basis of Therapeutics, 10th Edition, A. Gilman, J. Hardman and L. Limbird, eds., McGraw-Hill Press, 155-173, 2001), which is incorporated herein by reference in its entirety.

Compounds of formula (I), and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present disclosure is directed to methods of treating diseases or disorders by reducing the level or activity of HDAC3. The methods entail administration of a therapeutically effective amount of a compound formula (I), or a pharmaceutically acceptable salt or stereoisomer thereof, to a subject in need thereof.

The diseases or disorders are characterized or mediated by aberrant HDAC3 activity (e.g., elevated levels of HDAC3 or otherwise functionally abnormal HDAC3, e.g., mutant HDAC3 activity, relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary types of non-cancerous diseases or disorders that may be amenable to treatment with the compounds of the present disclosure include neurodegenerative diseases and autoimmune diseases.

As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the neurodegenerative disease is Parkinson's disease, Alzheimer's disease, or Huntington's disease.

As used herein, the term “autoimmune disease” refers to the condition where the immune system produces antibodies that attack normal body tissues. Representative examples of autoimmune diseases include Sjogren's syndrome, Hashimoto thyroiditis, rheumatoid arthritis, juvenile (type 1) diabetes, polymyositis, scleroderma, Addison disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, and temporal arteritis.

In some embodiments, compounds of formula (I) may be useful in the treatment of cell proliferative diseases and disorders (e.g., cancer). As used herein, the term “cell proliferative disease or disorder” refers to the conditions characterized by deregulated or abnormal cell growth, or both, including noncancerous conditions such as neoplasms, precancerous conditions, benign tumors, and cancer.

In some embodiments, methods of the present disclosure entail treatment of subjects having cell proliferative diseases or disorders of the hematological system, liver, lungs, pancreas, prostate, and breasts.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms, and TP63 rearranged lymphoma.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, methods of the present disclosure may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

In some embodiments, the cancer is laryngeal cancer, neuroblastoma, Sezary syndrome, or glioblastoma multiforme, or non-small-cell lung carcinoma.

### Pharmaceutical Kits

The present compounds and their pharmaceutically acceptable salts and stereoisomers and/or compositions containing them may be assembled into kits or pharmaceutical systems. Kits or pharmaceutical systems according to this aspect of the disclosure include a carrier or package such as a box, carton, tube or the like, having in close confinement therein one or more containers, such as vials, tubes, ampoules, or bottles, which contain a compound of formula (I), or a pharmaceutical composition thereof. The kits or pharmaceutical systems of the disclosure may also include printed instructions for using the compounds and compositions.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1: General Methods

Unless otherwise noted, reagents and solvents were used as received from commercial suppliers. All reactions were monitored using an Agilent LC/MS system (Agilent 1200LC/G6130A MS) using SunFire™ C18 column (4.6×50 mm, 3.5 μm particle size), LC method: solvent gradient=95% A to 5% A; solvent A=0.01% TFA in Water; solvent B=0.01% TFA in ACN; flow rate: 2.0 mL/min, column temperature 50° C. Purification of reaction products was carried out with flash chromatography using CombiFlash® Rf with Teledyne Isco RediSep® normal-phase silica flash columns; or Gilson 281 (PHG008) using Welch C18 column (21.2×250 mm, 10 μm particle size): solvent gradient 10% to 95% acetonitrile in H₂O (0.05% TFA as additive); flow rate: 30 mL/min. The purity of all compounds was over 95% and was analyzed with Agilent LC/MS system. 1H NMR spectra were obtained using Bruker Avance III spectrometers (400 MHz for 1H). Chemical shifts are reported relative to dimethyl sulfoxide (δ=2.50) for 1H NMR. Spectra are given in ppm (δ) and as br=broad, s=singlet, d=doublet, t=triplet, q=quartet, m=multiplet and coupling constants J are reported in Hertz.

### Example 2: Synthesis of (2S,4R)-1-((S)-14-(tert-butyl)-1-(1H-indol-3-yl)-12-oxo-3-(4-((E)-3-oxo-3-(2-propylhydrazineyl)prop-1-en-1-yl)benzyl)-6,9-dioxa-3,13-diazapentadecan-15-oyl)-4-hydroxy-N—((S)-1-(4-(4-methylthiazol-5-yl)phenyl)ethyl) pyrrolidine-2-carboxamide (2)

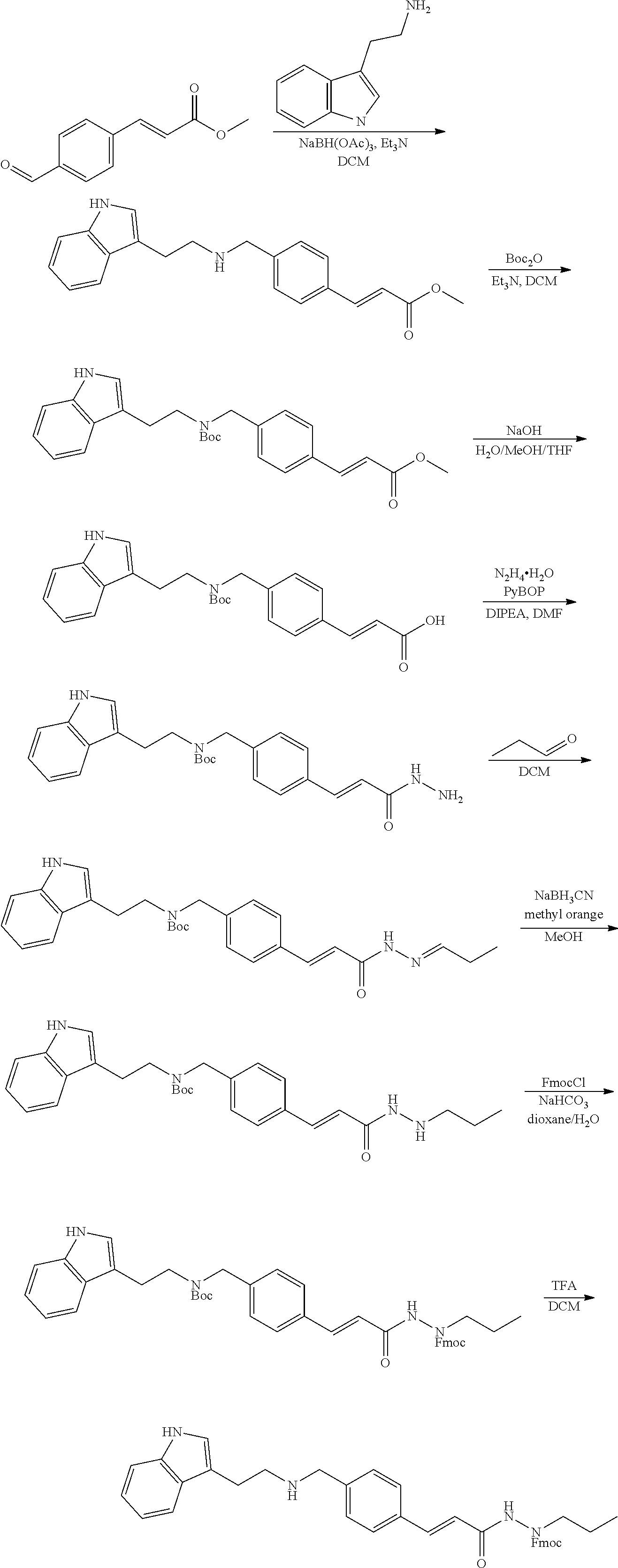

### Methyl (E)-3-(4-(((2-(1H-indol-3-yl)ethyl)amino)methyl)phenyl) acrylate

To a mixture of tryptamine (8.42 g, 52.604 mmol) and Et₃N (26.67 g, 263.021 mmol) in dichloromethane (100 mL), a solution of methyl (E)-3-(4-formylphenyl) acrylate (5.01 g, 26.302 mmol) in dichloromethane (20 mL) was added dropwise at room temperature. The reaction mixture was stirred at room temperature for 1.5 h, and then NaBH(AcO)₃ (22.29 g, 105.208 mmol) was added over a 20 min period at room temperature. The resulting reaction mixture was stirred at room temperature overnight, and quenched slowly by addition of saturated NaHCO₃ solution (50 mL), diluted with H₂O (300 mL) and extracted with dichloromethane (3×100 mL). The combined organic layer was washed with brine (100 mL), dried over anhydrous Na₂SO₄ and concentrated in vacuo. The residue was purified with column chromatography on silica gel (PE/EtOAc, 65% to 100%, with 0.2% Et₃N) to give the title compound as a yellow solid (6.61 g, 75.1% yield). LC-MS Mass m/z: 335.0 [M+H]⁺.

### Methyl (E)-3-(4-(((2-(1H-indol-3-yl)ethyl) (tert-butoxycarbonyl)amino)methyl)phenyl) acrylate

To a mixture of methyl (E)-3-(4-(((2-(1H-indol-3-yl)ethyl)amino)methyl)phenyl) acrylate (7.51 g, 22.476 mmol) and Et₃N (6.82 g, 67.415 mmol) in dichloromethane (80 mL), Boc₂O (9.81 g, 44.943 mmol) was added at room temperature. The reaction mixture was stirred at room temperature for 3 h, and concentrated in vacuo. The residue was purified with column chromatography on silica gel (PE/EtOAc, 0% to 50%, with 0.1% Et₃N) to give the title compound as a yellow oil (8.01 g, 82.0% yield). LC-MS Mass m/z: 335.0 [M−Boc+H]⁺, 379.0 [M−56+H]⁺.

### (E)-3-(4-(((2-(1H-indol-3-yl)ethyl) (tert-butoxycarbonyl)amino)methyl)phenyl) acrylic acid

A mixture of methyl (E)-3-(4-(((2-(1H-indol-3-yl)ethyl) (tert-butoxycarbonyl)amino)methyl)phenyl) acrylate (8.01 g, 18.447 mmol), NaOH (2.21 g, 55.342 mmol), H₂O (20 mL) and MeOH (20 mL) in THF (60 mL) was stirred at room temperature for 3 h. The reaction mixture was concentrated in vacuo, and diluted with H₂O (100 mL), and the pH was adjusted to 5 using 1 N aqueous HCl. The residue was extracted with EtOAc (3×35 mL) and the combined organic layer was dried over anhydrous Na₂SO₄ and concentrated in vacuo to give the title compound as light yellow solid (6.38 g, 82.3%). LC-MS Mass m/z: 320.9 [M−Boc+H]⁺, 364.9 [M−56+H]⁺.

### tert-Butyl (E)-(2-(1H-indol-3-yl)ethyl) (4-(3-hydrazineyl-3-oxoprop-1-en-1-yl)benzyl) carbamate

To a mixture of (E)-3-(4-(((2-(1H-indol-3-yl)ethyl) (tert-butoxycarbonyl)amino)methyl)phenyl) acrylic acid (6.01 g, 14.279 mmol), DIPEA (7.28 g, 57.116 mmol) and PyBOP (10.41 g, 19.991 mmol) in DMF (1.0 mL), N₂H₄: H₂O (0.79 g, 18.562 mmol) was added dropwise at room temperature. The reaction mixture was stirred at room temperature overnight, diluted with saturated NaHCO₃ solution (150 mL) and extracted with EtOAc (3×40 mL). The combined organic layer was washed with brine (50 mL), dried over anhydrous Na₂SO₄ and concentrated in vacuo. The residue was purified with preparative HPLC (CH₃CN/H₂O, 0% to 70%, with 0.1% TFA) to give the title compound as a yellow oil (7.51 g, crude). LC-MS Mass m/z: 378.9 [M−56+H], 335.0 [M−100+H]⁺.

### tert-Butyl(2-(1H-indol-3-yl)ethyl) (4-((E)-3-oxo-3-(2-((E)-propylidene) hydrazineyl) prop-1-en-1-yl)benzyl) carbamate

A mixture of tert-butyl (E)-(2-(1H-indol-3-yl)ethyl) (4-(3-hydrazineyl-3-oxoprop-1-en-1-yl)benzyl) carbamate (7.40 g, 12.437 mmol) and propionaldehyde (0.87 g, 14.923 mmol) in dichloromethane (40 mL) was stirred at room temperature for 3 h. The reaction mixture was concentrated in vacuo to give the title compound as a yellow oi (6.31 g, crude). LC-MS Mass m/z: 474.9 [M+H]⁺, 418.9 [M−56+H]⁺.

### tert-Butyl (E)-(2-(1H-indol-3-yl)ethyl) (4-(3-oxo-3-(2-propylhydrazineyl) prop-1-en-1-yl)benzyl) carbamate

To a mixture of tert-butyl(2-(1H-indol-3-yl)ethyl) (4-((E)-3-oxo-3-(2-((E)-propylidene) hydrazineyl) prop-1-en-1-yl)benzyl) carbamate (6.31 g, 12.437 mmol, crude), methyl orange (0.05 g, pH indicator) and NaBH₃CN (2.32 g, 37.311 mmol) in MeOH (40 mL) was added an HCl solution in MeOH (2 M, about 15 drops) dropwise to adjust pH to <3. The reaction mixture was stirred at room temperature for 3 h., quenched with saturated NaHCO₃ solution (10 mL) and H₂O (90 mL) and extracted with EtOAc (3×30 mL). The combined organic layer was dried over anhydrous Na₂SO₄ and concentrated in vacuo. The residue was purified with flash column chromatography on a C18 column (CH₃CN/H₂O, 0% to 80%) to give the title compound as a yellow oil (2.20 g, 37.1%). LC-MS Mass m/z: 477.0 [M+H]⁺.

### (9H-fluoren-9-yl)methyl (E)-2-(3-(4-(((2-(1H-indol-3-yl)ethyl) (tert-butoxycarbonyl)amino)methyl)phenyl) acryloyl)-1-propylhydrazine-1-carboxylate

To a mixture of tert-butyl (E)-(2-(1H-indol-3-yl)ethyl) (4-(3-oxo-3-(2-propylhydrazineyl) prop-1-en-1-yl)benzyl) carbamate (1650 mg, 3.465 mmol), NaHCO₃ (1165 mg, 13.862 mmol) and H₂O (25 mL) in 1,4-dioxane (20 mL), a solution of FmocCl (1789 mg, 6.931 mmol) in 1,4-dioxane (5.0 mL) was added dropwise at room temperature. The reaction mixture was stirred at room temperature overnight. The reaction mixture was diluted with H₂O (80 mL) and extracted with EtOAc (3×20 mL). The combined organic layer was dried over anhydrous Na₂SO₄ and concentrated in vacuo. The residue was purified with flash column chromatography on a C18 column (CH₃CN/H₂O, 0% to 80%, with 0.1% TFA) to give the title compound as a light yellow solid (1350 mg, 55.8% yield). LC-MS Mass m/z: 599.4 [M-Boc+H]⁺, 643.3 [M−56+H]⁺, 721.3 [M+Na]⁺.

### (9H-fluoren-9-yl)methyl (E)-2-(3-(4-(((2-(1H-indol-3-yl)ethyl)amino)methyl)phenyl) acryloyl)-1-propylhydrazine-1-carboxylate TFA salt

A mixture of (9H-fluoren-9-yl)methyl (E)-2-(3-(4-(((2-(1H-indol-3-yl)ethyl) (tert-butoxycarbonyl)amino)methyl)phenyl) acryloyl)-1-propylhydrazine-1-carboxylate (1.51 g, 2.162 mmol) and TFA (6.0 mL) in dichloromethane (6.0 mL) was stirred at room temperature for 3 h. The reaction mixture was concentrated in vacuo, and the residue was purified with flash column chromatography on a C18 column (CH₃CN/H₂O, 0% to 60%, with 0.1% TFA) to give the title compound as a white solid (1.05 g, 68.2%, TFA salt). LC-MS Mass m/z: 599.4 [M+H]⁺, 621.4 [M+Na]⁺.

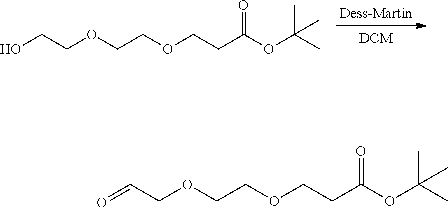 [SMILES: CC(C)(C)OC(=O)CCOCCOCCO ; CC(C)(C)OC(=O)CCOCCOCC=O]

### tert-Butyl 3-(2-(2-oxoethoxy) ethoxy) propanoate

A mixture of tert-butyl 3-(2-(2-hydroxyethoxy) ethoxy) propanoate (500 mg, 2.135 mmol) and Dess-Martin periodinane (1358 mg, 3.203 mmol) in dichloromethane (15 mL) was stirred at room temperature for 18 h. The reaction mixture was filtered, the solid residue was washed with EtOAc (5×5.0 mL) and the filtrate was concentrated in vacuo. The residue was diluted with saturated NaHCO₃ (70 mL) and extracted with EtOAc (3×20 mL). The combined organic layer was washed with saturated NaHCO₃ (2×20 mL), dried over anhydrous Na₂SO₄ and concentrated in vacuo to give the title compound as a colorless oil (360 mg, crude). LC-MS Mass m/z: 177.1 [M−56+H], 233.3 [M+H]⁺.

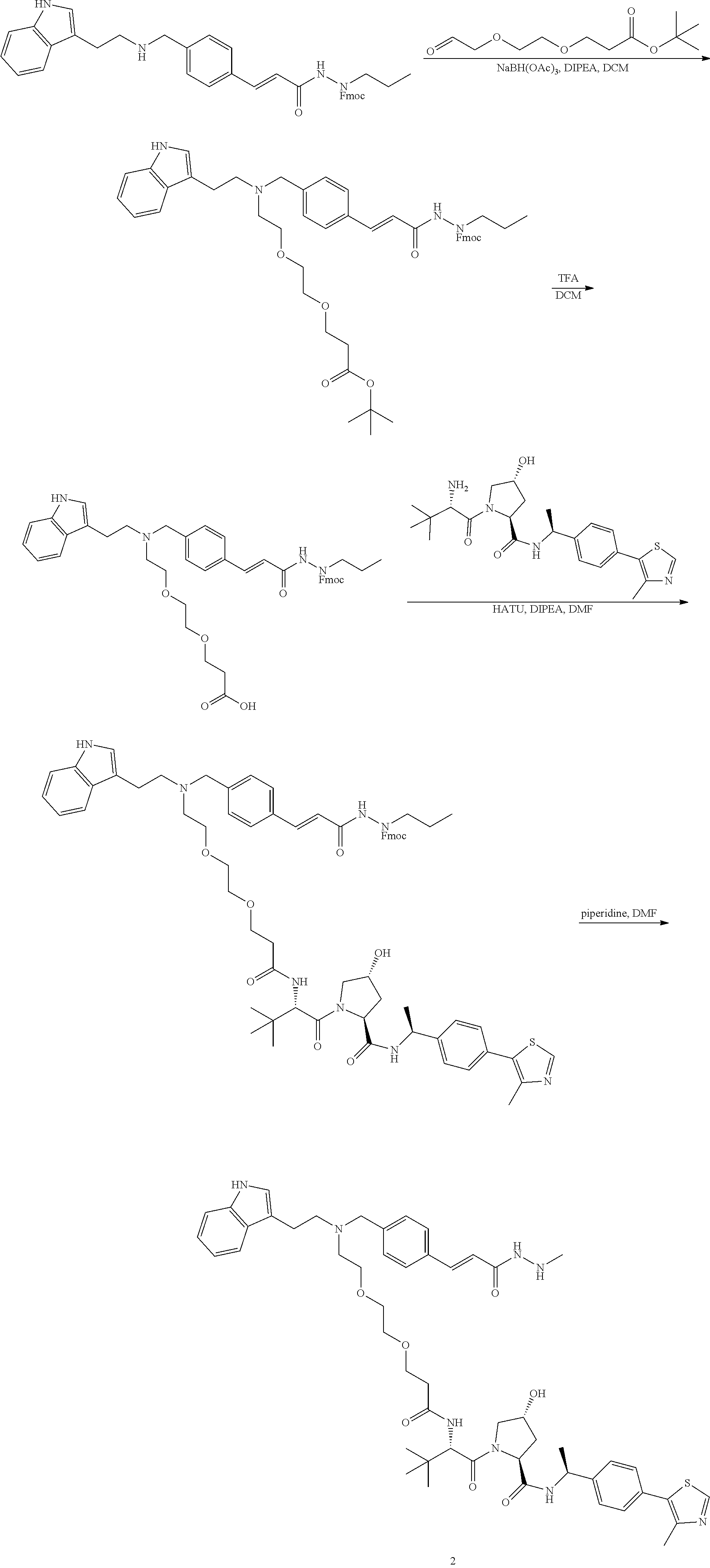

### (9H-fluoren-9-yl) methyl (E)-2-(3-(4-(2-(2-(1H-indol-3-yl)ethyl)-13,13-dimethyl-11-oxo-5,8,12-trioxa-2-azatetradecyl)phenyl)acryloyl)-1-propylhydrazine-1-carboxylate

A mixture of (9H-fluoren-9-yl) methyl (E)-2-(3-(4-(((2-(1H-indol-3-yl)ethyl)amino)methyl)phenyl) acryloyl)-1-propylhydrazine-1-carboxylate TFA salt (150 mg, 0.211 mmol), DIPEA (272 mg, 2.111 mmol) and tert-Butyl 3-(2-(2-oxoethoxy) ethoxy) propanoate (305 mg, 1.271 mmol, crude) in dichloromethane (10 mL) was stirred at room temperature for 0.5 h. To the above mixture was added NaBH(AcO)₃ (221 mg, 1.061 mmol). The reaction mixture was stirred at room temperature overnight. The mixture was diluted with H₂O (50 mL) and extracted with EtOAc (3×10 mL). The combined organic layer was dried over anhydrous Na₂SO₄ and concentrated in vacuo. The residue was purified with flash column chromatography on silica gel (PE/EtOAc, 0% to 80%, with 0.1% Et₃N) to give the title compound as a yellow oil (70 mg, 40.8%). LC-MS Mass m/z: 814.8 [M+H]⁺.

### (E)-3-(2-(2-((2-(1H-indol-3-yl)ethyl) (4-(3-(2-(((9H-fluoren-9-yl) methoxy) carbonyl)-2-propylhydrazineyl)-3-oxoprop-1-en-1-yl)benzyl)amino) ethoxy) ethoxy) propanoic acid, TFA salt

A mixture of (9H-fluoren-9-yl) methyl (E)-2-(3-(4-(2-(2-(1H-indol-3-yl)ethyl)-13,13-dimethyl-11-oxo-5,8, 12-trioxa-2-azatetradecyl)phenyl) acryloyl)-1-propylhydrazine-1-carboxylate (60 mg, 0.0738 mmol) and TFA (1.5 mL) in dichloromethane (1.5 mL) was stirred at room temperature for 3 h. The reaction mixture was concentrated in vacuo to give the title compound as a brown oil (70 mg, crude), which was used in the next step without further purification. LC-MS Mass m/z: 759.4 [M+H]⁺.

### (9H-fluoren-9-yl)methyl 2-((E)-3-(4-((S)-2-(2-(1H-indol-3-yl)ethyl)-13-((2S,4R)-4-hydroxy-2-(((S)-1-(4-(4-methylthiazol-5-yl)phenyl)ethyl) carbamoyl) pyrrolidine-1-carbonyl)-14,14-dimethyl-11-oxo-5,8-dioxa-2,12-diazapentadecyl)phenyl) acryloyl)-1-propylhydrazine-1-carboxylate

To a mixture of (E)-3-(2-(2-((2-(1H-indol-3-yl)ethyl) (4-(3-(2-(((9H-fluoren-9-yl) methoxy) carbonyl)-2-propylhydrazineyl)-3-oxoprop-1-en-1-yl)benzyl)amino) ethoxy) ethoxy) propanoic acid, TFA salt (70 mg, 0.0738 mmol, crude), DIPEA (77 mg, 0.590 mmol) and HATU (43 mg, 0.110 mmol) in DMF (1.5 mL), a solution of (2S,4R)-1-((S)-2-amino-3,3-dimethylbutanoyl)-4-hydroxy-N—((S)-1-(4-(4-methylthiazol-5-yl)phenyl)ethyl) pyrrolidine-2-carboxamide (35 mg, 0.0738 mmol) and DIPEA (19 mg, 0.148 mmol) in DMF (0.5 mL) was added at room temperature. The reaction mixture was stirred at room temperature for 18 h. The mixture was diluted with H₂O (50 mL) and extracted with EtOAc (3×10 mL). The combined organic layer was washed with brine (20 mL), dried over anhydrous Na₂SO₄ and concentrated in vacuo to give the title compound as a yellow oil (100 mg, crude) yellow oil. LC-MS Mass m/z: 593.0 [M/2+H]⁺, 1185.3 [M+H]⁺.

### (2S,4R)-1-((S)-14-(tert-butyl)-1-(1H-indol-3-yl)-12-oxo-3-(4-((E)-3-oxo-3-(2-propyl hydrazineyl) prop-1-en-1-yl)benzyl)-6,9-dioxa-3,13-diazapentadecan-15-oyl)-4-hydroxy-N—((S)-1-(4-(4-methylthiazol-5-yl)phenyl)ethyl) pyrrolidine-2-carboxamide (2)

A mixture of (9H-fluoren-9-yl)methyl 2-((E)-3-(4-((S)-2-(2-(1H-indol-3-yl)ethyl)-13-((2S,4R)-4-hydroxy-2-(((S)-1-(4-(4-methylthiazol-5-yl)phenyl)ethyl) carbamoyl) pyrrolidine-1-carbonyl)-14,14-dimethyl-11-oxo-5,8-dioxa-2,12-diazapentadecyl)phenyl) acryloyl)-1-propylhydrazine-1-carboxylate (90 mg, 0.0760 mmol) and piperidine (1.0 mL) in DMF (4.0 mL) was stirred at room temperature for 3 h. The reaction mixture was diluted with H₂O (50 mL) and extracted with EtOAc (3×10 mL). The combined organic layer was washed with brine (20 mL), dried over anhydrous Na₂SO₄ and concentrated in vacuo. The residue was purified with preparative HPLC (CH₃CN/H₂O, with 0.1% NH₄HCO₃) to give the title compound as a white solid (5.5 mg, 7.7%). LC-MS Mass m/z: 482.4 [M/2+H]⁺, 963.6 [M+H]⁺. ¹H NMR (500 MHz, DMSO-d₆) δ 10.96 (d, J=2.5 Hz, 1H), 10.62 (s, 1H), 9.77 (s, 1H), 8.98 (s, 1H), 8.36 (d, J=7.8 Hz, 1H), 7.85 (d, J=9.4 Hz, 1H), 7.72 (d, J=8.0 Hz, 2H), 7.64 (d, J=8.1 Hz, 2H), 7.60 (d, J=15.9 Hz, 1H), 7.46-7.40 (m, 3H), 7.39-7.34 (m, 3H), 7.21 (d, J=2.5 Hz, 1H), 7.08 (t, J=7.5 Hz, 1H), 6.97 (t, J=7.4 Hz, 1H), 6.68 (d, J=15.9 Hz, 1H), 4.90 (p, J=7.3 Hz, 1H), 4.54 (s, 2H), 4.52 (d, J=9.4 Hz, 1H), 4.41 (t, J=8.1 Hz, 1H), 4.28 (s, 1H), 3.89-3.79 (m, 2H), 3.74-3.28 (m, 12H), 3.25-3.09 (m, 2H), 2.91 (t, J=7.5 Hz, 2H), 2.55-2.47 (m, 1H), 2.45 (s, 3H), 2.33 (dt, J=14.8, 6.2 Hz, 1H), 2.06-1.98 (m, 1H), 1.79 (ddd, J=12.9, 8.6, 4.6 Hz, 1H), 1.54 (h, J=7.4 Hz, 2H), 1.36 (d, J=7.0 Hz, 3H), 0.94-0.88 (m, 12H).

### Example 3: Synthesis of (2S,4R)-1-((S)-2-(3-(2-((2-(1H-indol-3-yl)ethyl) (4-((E)-3-oxo-3-(2-propylhydrazineyl) prop-1-en-1-yl)benzyl)amino) ethoxy) propanamido)-3,3-dimethylbutanoyl)-4-hydroxy-N—((S)-1-(4-(4-methylthiazol-5-yl)phenyl)ethyl) pyrrolidine-2-carboxamide (1)

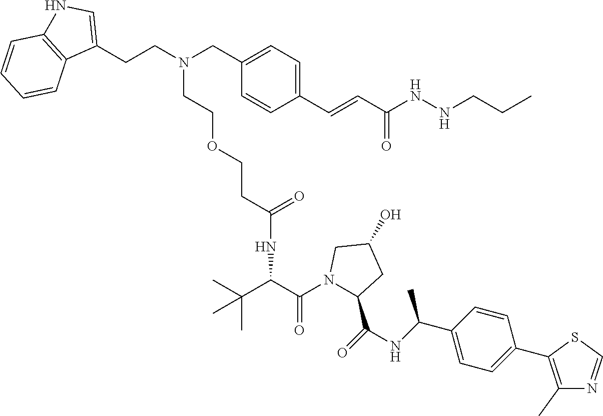 [SMILES: CCCNNC(=O)/C=C/c1ccc(CN(CCOCCC(=O)N[C@H](C(=O)N2C[C@H](O)C[C@H]2C(=O)N[C@@H](C)c2ccc(-c3scnc3C)cc2)C(C)(C)C)CCc2c[nH]c3ccccc23)cc1]

Compound 1 was synthesized from compound (9H-fluoren-9-yl) methyl (E)-2-(3-(4-(((2-(1H-indol-3-yl)ethyl)amino)methyl)phenyl) acryloyl)-1-propylhydrazine-1-carboxylate and tert-butyl 3-(2-oxoethoxy) propanoate using similar method compound 2 in Example 2. LC-MS Mass m/z: 459.9 [M/2+H]⁺, 919.7 [M+Na]⁺. 1H NMR (400 MHz, CD₃OD): δ (ppm) 8.86 (s, 1H), 7.73-7.48 (m, 4H), 7.47-7.27 (m, 8H), 7.11-6.99 (m, 2H), 6.93 (t, J=7.2 Hz, 1H), 6.56 (t, J=1.6 Hz, 1H), 4.62-4.40 (m, 6H), 3.90-3.53 (m, 8H), 2.98-2.78 (m, 6H), 2.57-2.31 (m, 6H), 2.17 (t, J=9.2 Hz, 1H), 1.99-1.86 (m, 1H), 1.65-1.50 (m, 2H), 1.47 (t, J=10.2 Hz, 3H), 1.18-0.95 (m, 11H).

### Example 4: Synthesis of (2S,4R)—N-(2-((5-((2-(1H-indol-3-yl)ethyl) (4-((E)-3-oxo-3-(2-propylhydrazineyl) prop-1-en-1-yl)benzyl)amino) pentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (3)

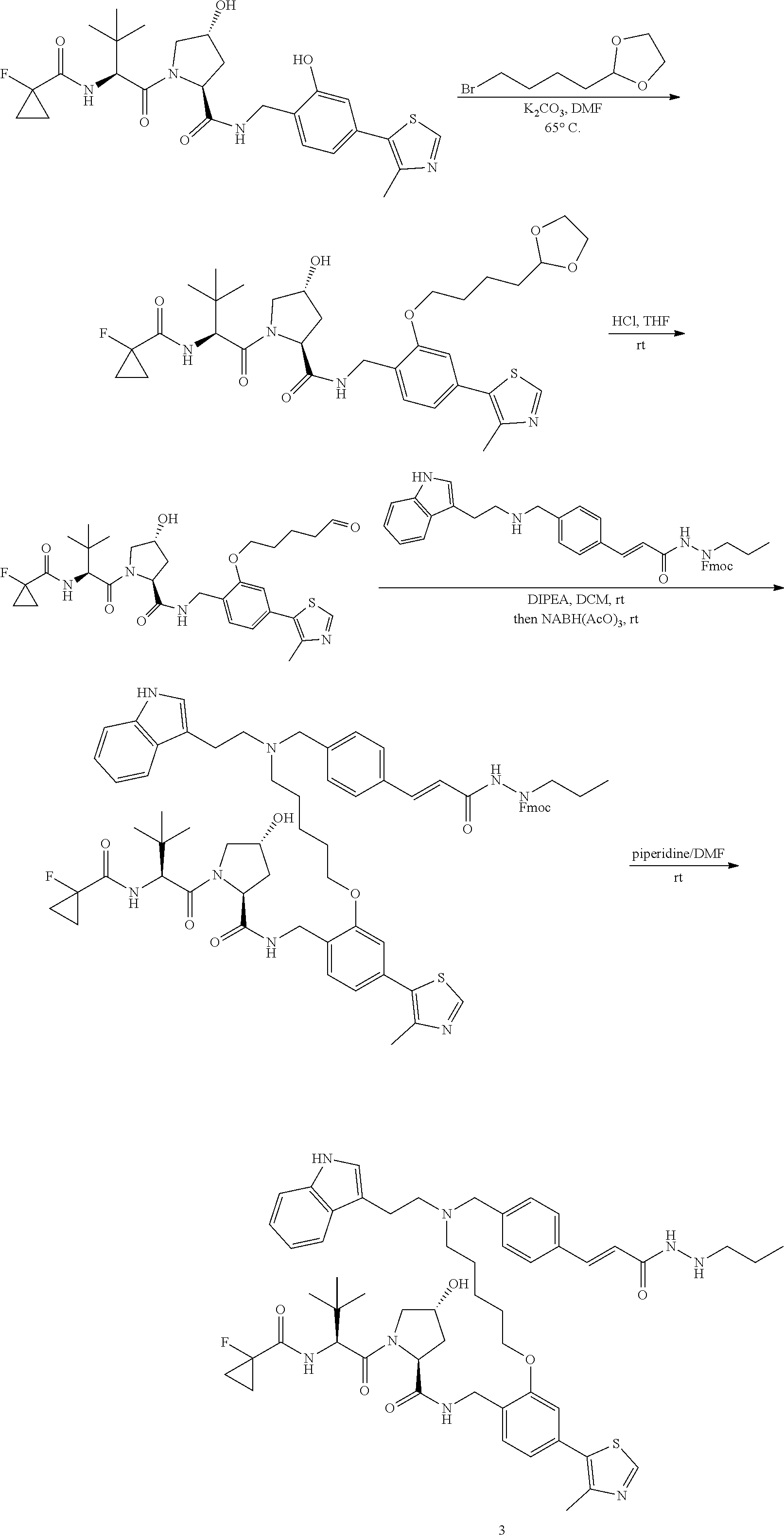

### (2S,4R)—N-(2-(4-(1,3-dioxolan-2-yl) butoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

A mixture of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl) pyrrolidine-2-carboxamide (150 mg, 0.282 mmol), 2-(4-bromobutyl)-1,3-dioxolane (70 mg, 0.338 mmol) and K₂CO₃ (58 mg, 0.423 mmol) in DMF (3.0 mL) was stirred at 65° C. for 5 hours. The reaction mixture was diluted with H₂O (50 mL) and extracted with EtOAc (3×20 mL). The combined organic layer was washed with brine (1×50 mL), dried over anhydrous Na₂SO₄ and concentrated in vacuo. The residue was purified with flash column chromatography on silica gel (dichloromethane/MeOH, 0%-15%) to give the title compound as a yellow oil (170 mg, 91.3%). LC-MS Mass m/z: 660.7 [M+H]⁺.

### (2S,4R)-1-((S)-2-(1-fluorocyclopropane-1-carboxamido)-3,3-dimethylbutanoyl)-4-hydroxy-N-(4-(4-methylthiazol-5-yl)-2-((5-oxopentyl)oxy)benzyl) pyrrolidine-2-carboxamide

A mixture of (2S,4R)—N-(2-(4-(1,3-dioxolan-2-yl) butoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (65 mg, 0.0968 mmol) and HCl (2.0 mL, 2 N in H₂O) in THF (2.0 mL) was stirred at room temperature for 3 h. The reaction mixture was quenched by addition of saturated NaHCO₃ solution (50 mL) and extracted with EtOAc (3×20 mL). The combined organic layer was dried over anhydrous Na₂SO₄ and concentrated in vacuo to give the title compound (80 mg, crude), which was used in the next step without further purification. LC-MS Mass m/z: 617.4 [M+H]⁺, 639.3 [M+Na]⁺.

### (9H-fluoren-9-yl)methyl 2-((E)-3-(4-(((2-(1H-indol-3-yl)ethyl) (5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy) pentyl)amino)methyl)phenyl) acryloyl)-1-propylhydrazine-1-carboxylate

A mixture of (9H-fluoren-9-yl) methyl (E)-2-(3-(4-(((2-(1H-indol-3-yl)ethyl)amino)methyl)phenyl) acryloyl)-1-propylhydrazine-1-carboxylate (105 mg, 0.129 mmol, crude), DIPEA (125 mg, 0.968 mmol) and (2S,4R)-1-((S)-2-(1-fluorocyclopropane-1-carboxamido)-3,3-dimethylbutanoyl)-4-hydroxy-N-(4-(4-methylthiazol-5-yl)-2-((5-oxopentyl)oxy)benzyl) pyrrolidine-2-carboxamide (80 mg, 0.0968 mmol) in dichloromethane (5.0 mL) was stirred at room temperature for 1.5 h. To the reaction mixture was added NaBH(AcO)₃ (82 mg, 0.387 mmol) at room temperature, and the reaction mixture was stirred at room temperature overnight. The reaction mixture was diluted with H₂O (50 mL) and extracted with EtOAc (3×10 mL). The combined organic layer was dried over anhydrous Na₂SO₄ and concentrated in vacuo. The residue was purified with flash column chromatography on silica gel (dichloromethane/MeOH, 0%-15%, basified with 0.1% Et₃N) to give the title compound as a yellow oil (75 mg, 51.6%). LC-MS Mass m/z: 600.5 [M/2+H]⁺, 1200.3 [M+H]⁺.

### (2S,4R)—N-(2-((5-((2-(1H-indol-3-yl)ethyl) (4-((E)-3-oxo-3-(2-propylhydrazineyl) prop-1-en-1-yl)benzyl)amino) pentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (3)

A mixture of (9H-fluoren-9-yl)methyl 2-((E)-3-(4-(((2-(1H-indol-3-yl)ethyl) (5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy) pentyl)amino)methyl)phenyl) acryloyl)-1-propylhydrazine-1-carboxylate (30 mg, 0.0250 mmol) and piperidine (0.6 mL) in DMF (2.4 mL) was stirred at room temperature for 3 h. The reaction mixture was diluted with H₂O (50 mL) and extracted with EtOAc (3×20 mL). The combined organic layer was washed with brine (1×50 mL), dried over anhydrous Na₂SO₄ and concentrated in vacuo. The residue was purified with preparative HPLC to give the title compound as a white solid (8.6 mg, 24.3%, TFA salt). LC-MS Mass m/z: 489.2 [M/2+H]⁺, 999.0 [M+Na]⁺. 1H NMR (400 MHz, DMSO-d₆): δ (ppm) 10.97 (s, 1H), 10.49 (brs, 1H), 9.73 (brs, 1H), 8.99 (s, 1H), 8.55 (d, J=6.0 Hz, 1H), 7.71 (d, J=8.2 Hz, 2H), 7.68-7.54 (m, 3H), 7.44-7.38 (m, 2H), 7.35 (d, J=8.1 Hz, 1H), 7.29 (dd, J=6.8, 2.4 Hz, 1H), 7.22 (d, J=2.1 Hz, 1H), 7.14-6.90 (m, 5H), 6.66 (d, J=15.8 Hz, 1H), 4.60 (d, J=9.2 Hz, 1H), 4.55-4.45 (m, 3H), 4.37-4.19 (m, 3H), 4.07 (d, J=4.8 Hz, 2H), 3.65-3.59 (m, 3H), 3.29-3.27 (m, 3H), 3.23-3.20 (m, 2H), 2.88 (t, J=4.8 Hz, 1H), 2.46 (s, 3H), 2.13-2.06 (m, 1H), 1.94-1.76 (m, 6H), 1.57-1.46 (m, 4H), 1.44-1.29 (m, 3H), 1.22 (d, J=8.4, 2.4 Hz, 2H), 0.96-0.89 (m, 12H). 19F-NMR (400 MHz, DMSO-d₆): −73.94, −196.20.

### Example 5: Cell Treatment and Protein Digestion

Kelly cells were treated with DMSO or 1 μM of compound 2 or 3 for 5 hr and cells were harvested by centrifugation at 4° C. before snap freezing in liquid nitrogen. Cell lysis was performed by the addition of Urea buffer (8 M Urea, 50 mM NaCl, 50 mM 4-(2-hydroxyethyl)-1-piperazineethanesulfonic acid (EPPS) pH 8.5, Protease and Phosphatase inhibitors) followed by manual homogenization by 20 passes through a 21-gauge (1.25 in. long) needle. Lysate was clarified by centrifugation and protein quantified using bradford (Bio-Rad) assay. 50 μg of protein for each sample was reduced, alkylated and precipitated using methanol/chloroform as described in Donovan et al., eLife 7: e38430 (2018). The resulting precipitated protein was resuspended in 4 M Urea, 50 mM HEPES pH 7.4, buffer for solubilization, followed by dilution to 1 M urea with the addition of 200 mM EPPS, pH 8. Proteins were digested for 12 hours at room temperature with LysC (1:50 ratio), followed by dilution to 0.5 M urea and a second digestion step was performed by addition of trypsin (1:50 ratio) for 6 hours at 37° C.

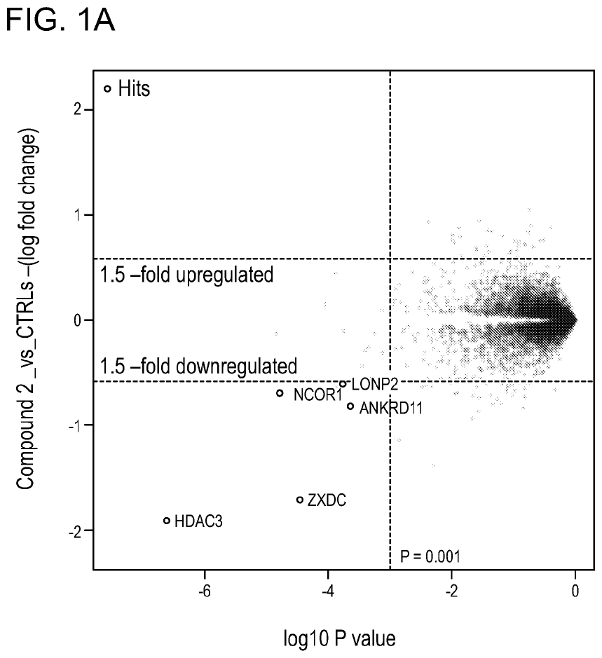

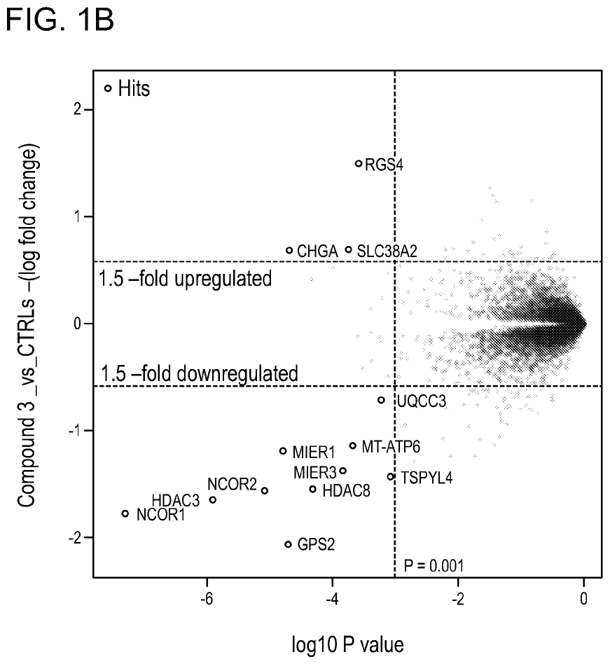

The results are summarized in the scatterplots illustrated in FIG. 1A and FIG. 1B. The scatterplots show the relative change in relative protein abundance in Kelly Cells with treatment of compounds 2 or 3.

### Example 6: Quantitative LCMS Proteomics

Sample digests were acidified with formic acid to a pH of 2-3 prior to desalting using C18 solid phase extraction plates (SOLA, Thermo Fisher Scientific™). Desalted peptides were dried in a vacuum-centrifuged and reconstituted in 0.1% formic acid for LC-MS analysis.

Data were collected using a TimsTOF Pro2 (Bruker Daltonics, Bremen, Germany) coupled to a nanoElute LC pump (Bruker Daltonics, Bremen, Germany) via a CaptiveSpray nano-electrospray source. Peptides were separated on a reversed-phase C18 column (25 cm×75 μm ID, 1.6 μM, IonOpticks, Australia) containing an integrated captive spray emitter. Peptides were separated using a 50 min gradient of 2-30% buffer B (acetonitrile in 0.1% formic acid) with a flow rate of 250 nL/min and column temperature maintained at 50° C.

DDA was performed in Parallel Accumulation-Serial Fragmentation (PASEF) mode to determine effective ion mobility windows for downstream diaPASEF data collection (Meier et al., Nature Methods 17:1229-1236 (2020)). The ddaPASEF parameters included: 100% duty cycle using accumulation and ramp times of 50 ms each, 1 TIMS-MS scan and 10 PASEF ramps per acquisition cycle. The TIMS-MS survey scan was acquired between 100-1700 m/z and 1/k0 of 0.7-1.3 V·s/cm². Precursors with 1-5 charges were selected and those that reached an intensity threshold of 20,000 arbitrary units were actively excluded for 0.4 min. The quadrupole isolation width was set to 2 m/z for m/z<700 and 3 m/z for m/z>800, with the m/z between 700-800 m/z being interpolated linearly. The TIMS elution voltages were calibrated linearly with three points (Agilent ESI-L Tuning Mix Ions; 622, 922, 1,222 m/z) to determine the reduced ion mobility coefficients (1/Ko). To perform diaPASEF, the precursor distribution in the DDA m/z-ion mobility plane was used to design an acquisition scheme for DIA data collection which included two windows in each 50 ms diaPASEF scan. Data was acquired using sixteen of these 25 Da precursor double window scans (creating 32 windows) which covered the diagonal scan line for doubly and triply charged precursors, with singly charged precursors able to be excluded by their position in the m/z-ion mobility plane. These precursor isolation windows were defined between 400-1200 m/z and 1/k0 of 0.7-1.3 V·s/cm².

### Example 7: DiaPASEF LC-MS Data Analysis

The diaPASEF raw file processing and controlling peptide and protein level false discovery rates, assembling proteins from peptides, and protein quantification from peptides was performed using library free analysis in DIA-NN 1.8 (Demichev et al., Nature Methods 17:41-44 (2020)). Library free mode performs an in silico digestion of a given protein sequence database alongside deep learning-based predictions to extract the DIA precursor data into a collection of MS2 spectra. The search results are then used to generate a spectral library which is then employed for the targeted analysis of the DIA data searched against a Swissprot human database (January 2021). Database search criteria largely followed the default settings for directDIA including: tryptic with two missed cleavages, carbomidomethylation of cysteine, and oxidation of methionine and precursor Q-value (FDR) cut-off of 0.01. Precursor quantification strategy was set to Robust LC (high accuracy) with RT-dependent cross run normalization. Proteins with missing values in any of the treatments and with poor quality data were excluded from further analysis (summed abundance across channels of <100 and mean number of precursors used for quantification<2). Protein abundances were scaled using in-house scripts in the R framework (R Core Team, A language and environment for statistical computing, 2014) and statistical analysis was carried out using the limma package within the R framework (Ritchie et al., Nucleic Acids Res. 43: e47 (2015)).

The scatterplots in FIG. 1A-FIG. 1B show the relative change in relative protein abundance with treatment of Kelly cells with compounds 2 (FIG. 1A) and 3 (FIG. 1B) compared to dimethyl sulfoxide (DMSO) control. Significant changes were assessed by moderated t-test and displayed with log 2 fold change on the y-axis and negative log 10 P values on the x-axis for one independent biological replicate of the compound and three independent biological replicates of DMSO. As shown, treatment with compounds 2 and 3 induced a significant and selective reduction in HDAC3 protein levels when compared to the DMSO treated cells.

### Example 8: Cellular VHL Engagement Assay

Cells stably expressing the BRD4BD2-GFP with mCherry reporter were seeded at a density of 1000-4000 cells/well in 384-well plates with 50 μL per well of FluoroBrite™ DMEM media (Thermo Fisher Scientific™, A18967) supplemented with 2% FBS a day before compound treatment. Compounds 1 and 2, and 250 nM 1 ((2S,4R)-1-(2R)-2-acetamido-3-[[6-[2-[(6S)-4-(4-chlorophenyl)-2,3,9-trimethyl-6H-thieno[3,2-f][1,2,4]triazolo[4,3-a][1,4]diazepin-6-yl]acetamido]hexyl]thio]-3-methylbutanoyl]-4-hydroxy-N-[4-(4-methylthiazol-5-yl)benzyl) pyrrolidinine-2-carboxamide) were dispensed using a D300e Digital Dispenser (HP), normalized to 0.5% DMSO, and incubated with the cells for 5 h. The assay plate was imaged immediately using an Acumen® High Content Imager (TTP Labtech) with 488 nm and 561 nm lasers in a 2 μm×1 μm grid per well format. The resulting images were analyzed using CellProfiler™.

A series of image analysis steps (an ‘image analysis pipeline’) was constructed. First, the red and green channels were aligned and cropped to target the middle of each well (to avoid analysis of the heavily clumped cells at the edges). A background illumination function was calculated for both red and green channels of each well individually and subtracted to correct for illumination variations across the 384-well plate from various sources of error. An additional step was then applied to the green channel to suppress the analysis of large auto fluorescent artifacts and enhance the analysis of cell specific fluorescence by way of selecting for objects under a given size (30 A.U.) and with a given shape (speckles). mCherry-positive cells were then identified in the red channel by filtering for objects 8-60 pixels in diameter and by using intensity to distinguish between clumped objects. The green channel was then segmented into GFP positive and negative areas and objects were labeled as GFP positive if at least 40% of it overlapped with a GFP positive area. The fraction of GFP-positive cells/mCherry-positive cells in each well was then calculated, and the green and red images were rescaled for visualization. The values for the concentrations that lead to a 50% increase in BRD4BD2-eGFP accumulation (EC₅₀) were calculated using the nonlinear fit variable slope model (GraphPad Software).

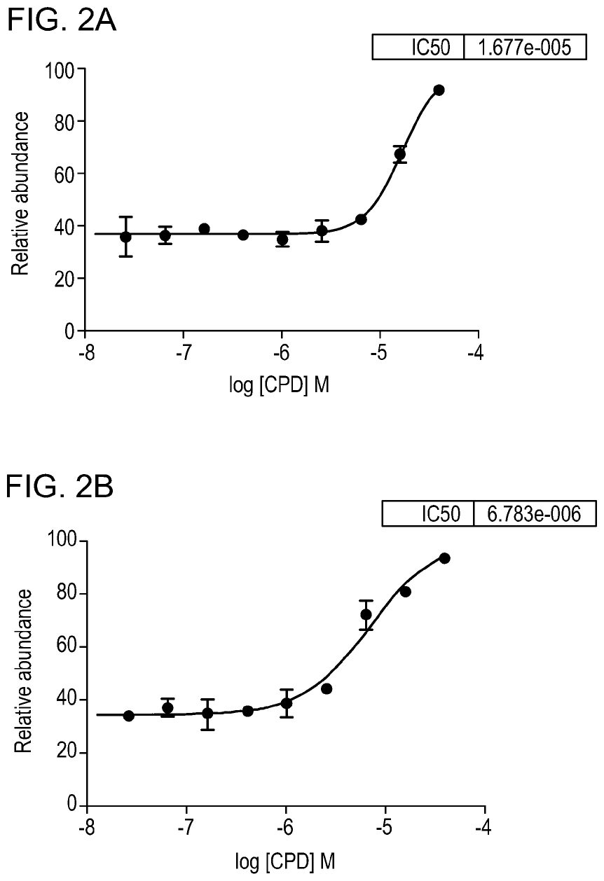

The results of the cellular VHL engagement assay are illustrated in FIG. 2A and FIG. 2B. They show that compounds 1 and 2 exhibited IC₅₀ values of 16.8 μM and 6.78 μM respectively.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A compound of formula (I): 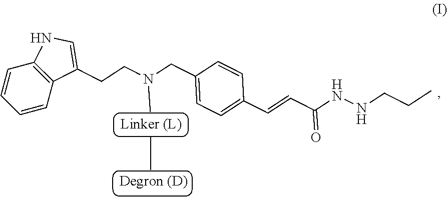
or a pharmaceutically acceptable salt or stereoisomer thereof,
wherein:
the Linker represents a moiety that connects covalently the nitrogen atom and Degron;
and
the Degron is of Formula D1 or D2: 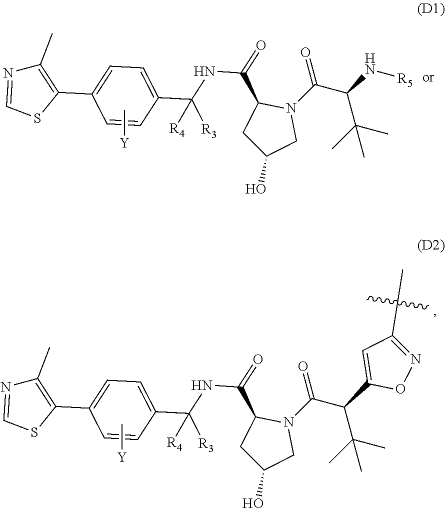
wherein:
R₃ is hydrogen or optionally substituted C₁-C₃ alkyl, or
R₃ and R₄, together with the carbon atom to which they are attached, form cyclopropyl;
R₄ is hydrogen, methyl, or 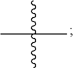
R₅ is C(O)CR₆R₇R₈, 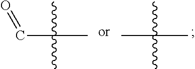
R₆ and R₇ are hydrogen, or
R₆ and R₇, together with the carbon atom to which they are attached, form cyclopropyl;
R₈ is hydrogen, fluoro, cyano, or NMe₂; and
Y is hydrogen, 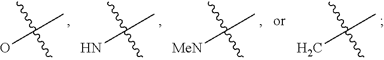
wherein 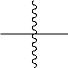
is a bond between the Degron and the Linker, provided that there is only one bond between the Degron and the Linker.

2. The compound of claim 1, wherein the Degron is of Formula D1.

3. The compound of claim 2, wherein R₃ is hydrogen and R₄ is

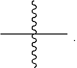

4. The compound of claim 3, wherein Formula D1 is of Formula D1a-D1d:

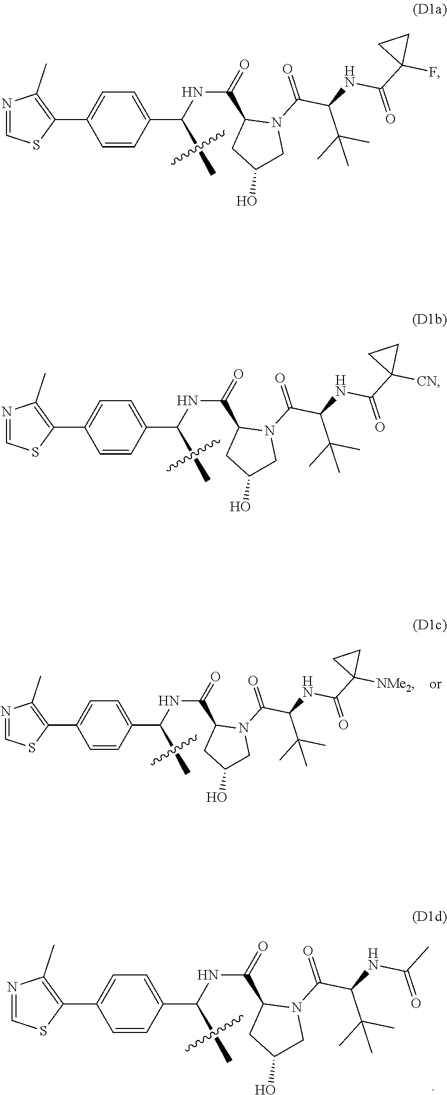

5. The compound of claim 2, wherein Y is

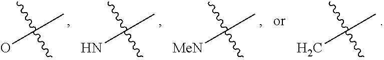

6. The compound of claim 5, wherein Formula D1 is of Formula D1e-D1h:

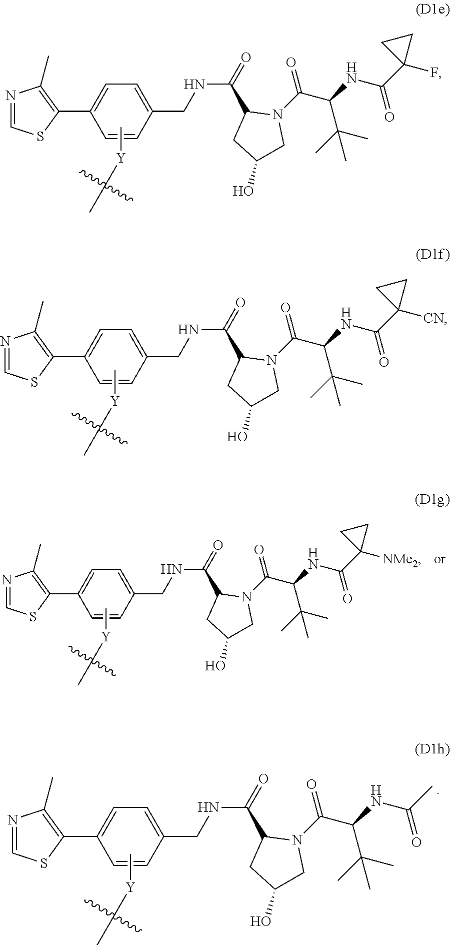

7. The compound of claim 2, wherein R₅ is

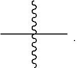

8. The compound of claim 7, wherein Formula D1 is of Formula D1i-D1o:

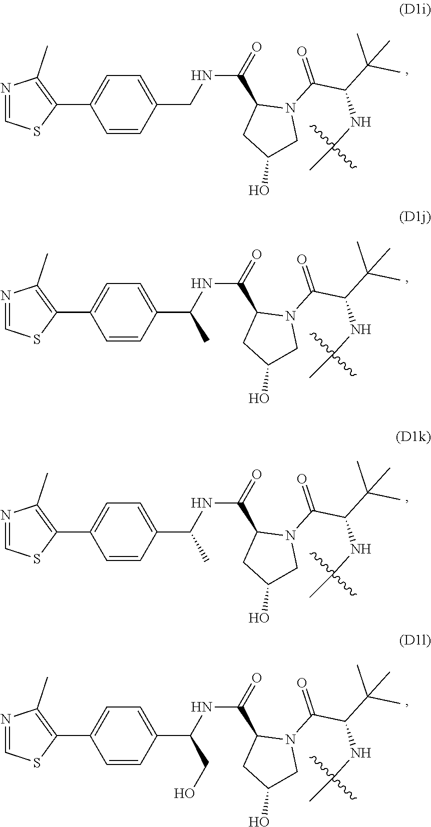 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)N[C@H](C)c1ccc(-c2scnc2C)cc1)C(C)(C)C ; *N[C@H](C(=O)N1C[C@H](O)C[C@H]1C(=O)N[C@@H](CO)c1ccc(-c2scnc2C)cc1)C(C)(C)C]

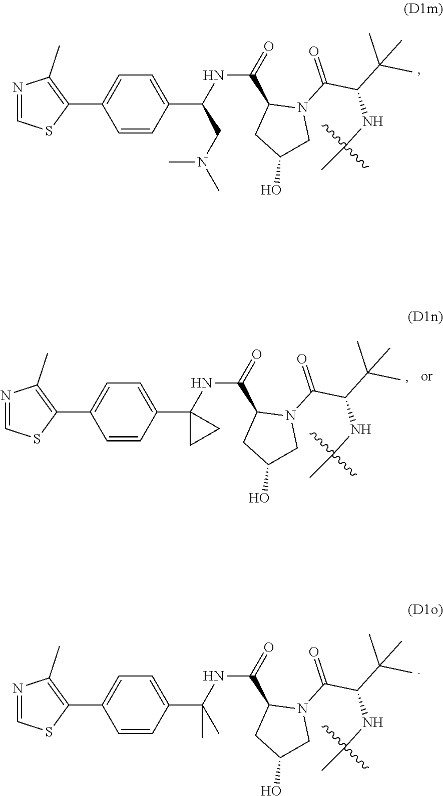 [SMILES: *N[C@H](C(=O)N1C[C@H](O)C[C@H]1C(=O)N[C@@H](CN(C)C)c1ccc(-c2scnc2C)cc1)C(C)(C)C ; *N[C@H](C(=O)N1C[C@H](O)C[C@H]1C(=O)NC1(c2ccc(-c3scnc3C)cc2)CC1)C(C)(C)C ; *N[C@H](C(=O)N1C[C@H](O)C[C@H]1C(=O)NC(C)(C)c1ccc(-c2scnc2C)cc1)C(C)(C)C]

9. The compound of claim 1, wherein the Degron is of Formula D2.

10. The compound of claim 9, wherein Formula D2 is of Formula D2a-D2g:

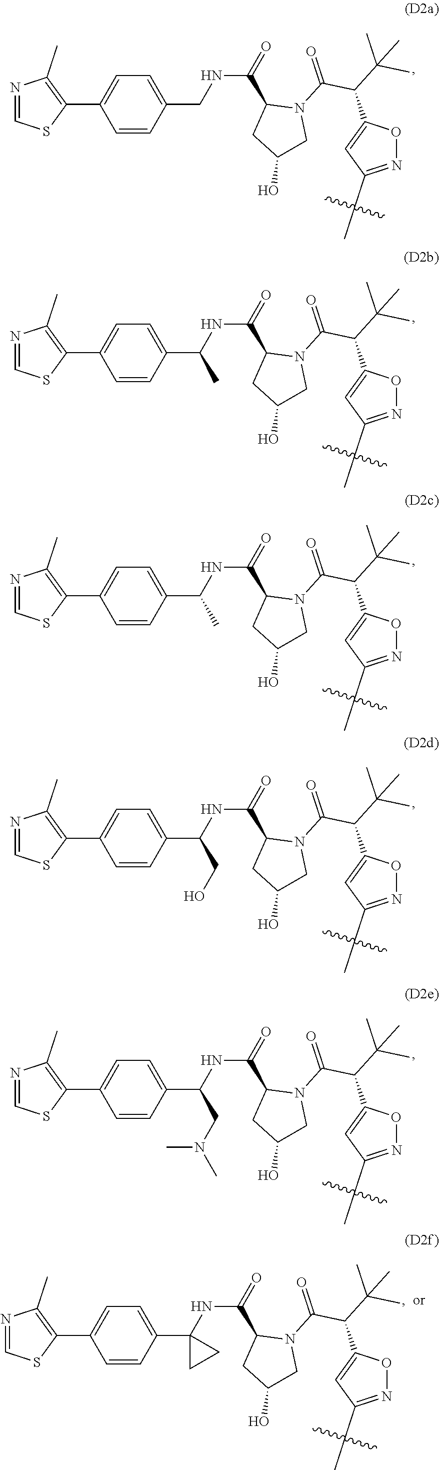

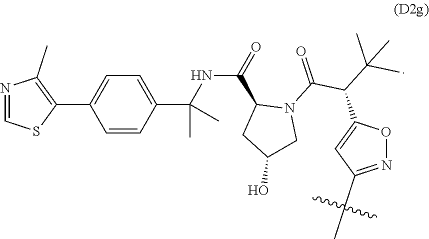 [SMILES: *c1cc([C@H](C(=O)N2C[C@H](O)C[C@H]2C(=O)NC(C)(C)c2ccc(-c3scnc3C)cc2)C(C)(C)C)on1]

11. The compound of claim 1, wherein the Linker is a bond or comprises an alkylene chain or a bivalent alkylene chain, either of which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different, or
wherein the Linker is a polyethylene glycol (PEG) chain which may be interrupted by, and/or terminates at either or both termini with at least one of —O—, —S—, —N(R′)—, —C≡C—, —C(O)—, —C(O)O—, —OC(O)—, —OC(O)O—, —C(NOR′)—, —C(O)N(R′)—C(O)N(R′)C(O)—, —C(O)N(R′)C(O)N(R′)—, —N(R′)C(O)—, —N(R′)C(O)N(R′)—N(R′) C(O)O—, —OC(O)N(R′)—, —C(NR′)—, —N(R′)C(NR′)—, —C(NR′)N(R′)—, N(R′)C(NR′)N(R′)—, —OB (Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

12. The compound of claim 11, wherein the alkylene chain contains 2 to 15 alkylene units, or
wherein the PEG chain contains 1 to 5 PEG units.

13. (canceled)

14. (canceled)

15. The compound of claim 1, wherein the Linker is of Formula L0: 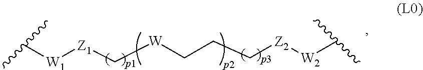
or stereoisomer thereof, wherein
p1 is an integer selected from 0 to 6;
p2 is an integer selected from 0 to 12;
p3 is an integer selected from 0 to 12;
each W is independently absent, CH₂, O, S, NR₁₀, or C(O) NR₁₀;
each R₁₀ is independently hydrogen or C₁-C₆ alkyl;
W₁ and W₂ are independently absent, (CH₂)₁₋₃, O, or NH; and
Z₁ and Z₂ are independently absent, —O—, —S—, —N(R₁₀)—, —C≡C—, —C(O)—, —C(O)O—, —OC(O)—, —OC(O)O—, —C(NOR₁₀)—, —C(O)N(R₁₀)—, —C(O)N(R₁₀)C(O)—, —C(O)N(R₁₀)C(O)N(R₁₀)—, —N(R₁₀)C(O)—, —N(R₁₀)C(O)N(R₁₀)—, —N(R₁₀)C(O)O—, —OC(O)N(R₁₀)—, —C(NR₁₀)—, —N(R₁₀)C(NR₁₀)—, —C(NR₁₀)N(R₁₀)—, —N(R₁₀)C(NR₁₀)N(R₁₀)—, —OB (Me)O—, —S(O)₂—, —OS(O)—, —S(O)O—, —S(O)—, —OS(O)₂—, —S(O)₂O—, —N(R₁₀)S(O)₂—, —S(O)₂N(R₁₀)—, —N(R₁₀) S(O)—, —S(O)N(R₁₀)—, —N(R₁₀)S(O)₂N(R₁₀)—, —N(R₁₀) S(O)N(R₁₀)—, C₃-C₁₂ carbocyclene, 3- to 12-membered heterocyclene, or 5- to 12-membered heteroarylene;
wherein the Linker is covalently bonded to the Degron via the 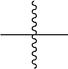
next to W₂, and covalently bonded to the nitrogen atom via the 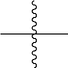
next to W₁, or the Linker is covalently bonded to the Degron via the 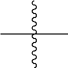
next to W₁, and covalently bonded to the nitrogen atom via the 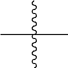
next to W₂.

16. The compound of claim 15, wherein formula L0 is of formula L0a-L0h: 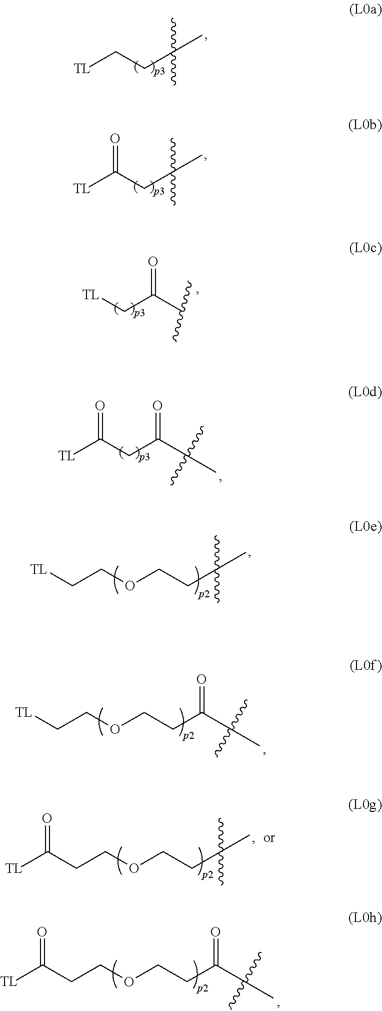
wherein TL represents targeting ligand.

17. The compound of claim 1, wherein the Linker is represented by any one of structures:

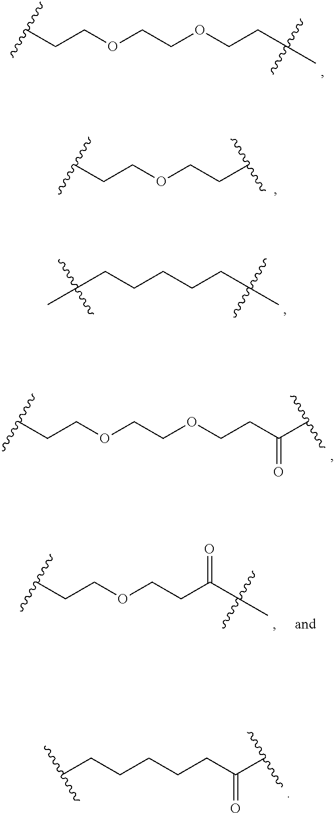 [SMILES: *CCOCCOCC* ; *CCOCC* ; *CCCCC* ; *CCOCCOCCC(*)=O ; *CCOCCC(*)=O ; *CCCCCC(*)=O]

18. The compound of claim 1, which is: 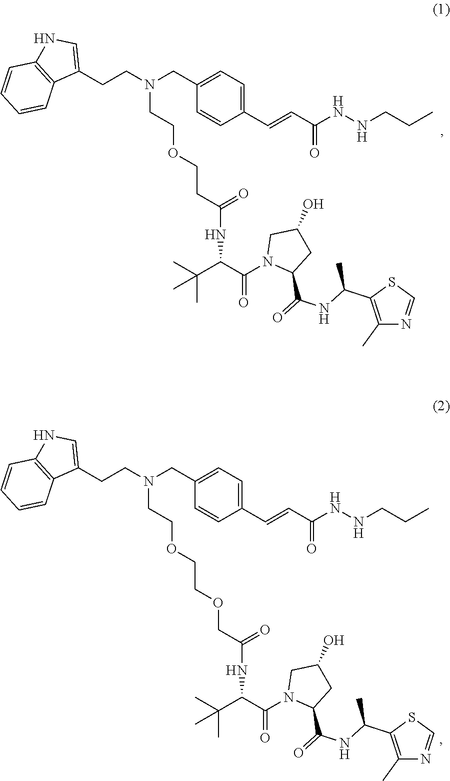 [SMILES: CCCNNC(=O)/C=C/c1ccc(CN(CCOCCC(=O)N[C@H](C(=O)N2C[C@H](O)C[C@H]2C(=O)N[C@@H](C)c2scnc2C)C(C)(C)C)CCc2c[nH]c3ccccc23)cc1 ; CCCNNC(=O)/C=C/c1ccc(CN(CCOCCOCC(=O)N[C@H](C(=O)N2C[C@H](O)C[C@H]2C(=O)N[C@@H](C)c2scnc2C)C(C)(C)C)CCc2c[nH]c3ccccc23)cc1]
or pharmaceutically acceptable salt or stereoisomer thereof.

19. A pharmaceutical composition, comprising a therapeutically effective amount of the compound or pharmaceutically acceptable salt or stereoisomer thereof of claim 1, and a pharmaceutically acceptable carrier.

20. A method of treating a disease or disorder characterized or mediated by aberrant HDAC3 activity, comprising administering a therapeutically effective amount of the compound of claim 1 or a pharmaceutically acceptable salt or stereoisomer thereof to a subject in need thereof.

21. The method of claim 20, wherein the disease or disorder is cancer, a neurodegenerative disease, or an autoimmune disease.

22. The method of claim 21, wherein the cancer is breast cancer, prostate cancer, pancreatic cancer, laryngeal cancer, lymphoma, neuroblastoma, polycythemia vera, T-cell lymphoma, multiple myeloma, leukemia, hepatocellular carcinoma, non-small cell lung cancer, essential thrombocythemia, Hodgkin's lymphoma, or TP63 rearranged lymphoma, or
wherein the neurodegenerative disease is Parkinson's disease, Alzheimer's disease, or Huntington's disease, or
wherein the autoimmune disease is Sjogren's syndrome, Hashimoto thyroiditis, rheumatoid arthritis, juvenile (type 1) diabetes, polymyositis, scleroderma, Addison disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, or temporal arteritis.

23. The method of claim 22, wherein the cancer is lymphoma, Hodgkin's lymphoma, or TP63 rearranged lymphoma.

24.-29. (canceled)
